(19) United States
(12) Patent Application Publication
(10) Pub. No.: US 2009/0182050 A1
(43) Pub. Date: Jul. 16, 2009

(54) Salts of Fatty Acids and Methods of Making and Using thereof

(75) Inventors: Colin Barrow, Nova Scotia (CA); Jaroslav A. Kralovec, Nova Scotia (CA)

Correspondence Address: Ballard Spahr Andrews & Ingersoll, LLP, SUITE 1000, 999 PEACHTREE STREET, ATLANTA, GA, 30309-3915, US

(73) Assignee: OCEAN NUTRITION CANADA, LTD.

(21) Appl. No.: 12/083,276

(22) PCT Filed: Oct. 3, 2006
(86) PCT No.: PCT/IB2006/004009

§ 371 Date: Feb. 10, 2009

Related U.S. Application Data
(60) Provisional application No. 60/724,644, filed on Oct. 7, 2005.
(60) Provisional application No. 60/775,664, filed on Feb. 22, 2006.

(51) Int. Cl. A61K 31/20; A61K 31/202; A61K 31/201

(52) U.S. Cl. 514/560

(57) ABSTRACT

Disclosed are methods of making salts of fatty acids (e.g., marine oils) and to salts prepared by the disclosed methods. Methods of using the disclosed salts are also disclosed.

### CROSS-REFERENCE TO RELATED APPLICATIONS

This application claims the benefit of priority to U.S. Provisional Application No. 60/724,644, filed Oct. 7, 2005, and U.S. Provisional Application No. 60/775,664, filed Feb. 22, 2006, which are both incorporated by reference herein in their entireties.

### FIELD

The disclosed matter relates to methods of making salts of fatty acids (e.g., marine oils) and to salts prepared by the disclosed methods. Methods of using the disclosed salts are also disclosed.

### BACKGROUND

Omega-3 fatty acids are vital to everyday life and function. For example, the beneficial effects of omega-3 fatty acids like cis-5,8,11,14,17-eicosapentaenoic acid (EPA) and cis-4,7,10,13,16,19-docosahexaenoic acid (DHA) on lowering serum triglycerides are well established. These compounds are also known for other cardioprotective benefits such as preventing cardiac arrhythmias, stabilizing atherosclerotic plaques, reducing platelet aggregation, and reducing blood pressure. See e.g., Dyrberg et al., In: Omega-3 Fatty Acids Prevention and Treatment of Vascular Disease. Kristensen et al., eds., Bi & Gi Publ., Verona-Springer-Verlag, London, pp. 217-26, 1995; O'Keefe and Harris, Am. J Cardiology 2000, 85:1239-41; Radack et al., “The effects of low doses of omega-3 fatty acid supplementation on blood pressure in hypertensive subjects: a randomized controlled trial.” Arch. Intern. Med. 1991, 151:1173-80; Harris, “Extending the cardiovascular benefits of omega-3 fatty acids.” Curr Atheroscler Rep 2005, 7:375-80; Holub, “Clinical nutrition: 4 omega-3 fatty acids in cardiovascular care.” CMAJ 2002, 166(5):608-15. Indeed, the American Heart Association has also reported that omega-3 fatty acids can reduce cardiovascular and heart disease risk. Other benefits of omega-3 fatty acids are those related to the prevention and/or treatment of inflammation and neurodegenerative diseases, and to improved cognitive development. See e.g., Sugano and Michihiro, “Balanced intake of polyunsaturated fatty acids for health benefits.” J. Oleo Sci. 2001, 50(5):305-11.

The fatty acids EPA and DHA can be synthesized in the human body from α-linolenic acid (18:3); however, the conversion rate from this precursor molecule is limited (Muskiet et al., “Is docosahexaenoic acid (DHA) essential? Lessons from DHA status regulation, our ancient diet, epidemiology and randomized controlled trials.” J. Nutr. 2004, 134(1):183-6). Accordingly, EPA and DHA in the body are primarily derived from dietary sources (e.g., oily fish). Diets rich in fish oils are known to have many beneficial effects for heart disease, cancer, arthritis, allergies, and other chronic diseases. Epidemiological clinical trials have shown that increasing the dietary intake of omega-3 fatty acids, in the form of fish or fish oil supplements, may reduce various risk factors associated with cardiovascular disease. See e.g., The American Heart Association, Scientific Statement, “Fish Consumption, Fish Oil, Omega-3 Fatty Acids and Cardiovascular Disease,” November 2002; Appel et al., “Does supplementation of diet with ‘fish oil’ reduce blood pressure? A meta-analysis of controlled clinical trials.” Arch. Intern. Med. 1993, 153(12):1429-1438; GISSI-Prevenzione Investigators. “Dietary supplementation with omega-3 polyunsaturated fatty acids and vitamin E after myocardial infarction: results of the GISSI-Prevenzione trial.” Lancet 1999, 354:447-55.

Despite the strong evidence for the benefit of omega-3 fatty acids like EPA and DHA in prevention of cardiovascular disease, the average daily consumption of these fatty acids by North Americans is estimated to be between 0.1 to 0.2 grams, compared to a suggested daily intake of 0.65 grams to confer benefit (Webb, “Alternative sources of omega-3 fatty acids.” Natural Foods Merchandiser 2005, XXVI(8):40-4). Since altering dietary patterns of populations is difficult and many people do not like to eat fish, dietary supplementation with EPA and DHA is an important approach to addressing this problem. Unfortunately, many supplements of omega-3 fatty acids are sensitive to oxidation and can be foul smelling and tasting. Further, compliance with dietary supplement regimens requires discipline, which is often wanting.

In light of the health benefits of omega-3 fatty acids, what is needed in the art are compositions that can provide the benefits of omega-3 fatty acids and which are stable and more palatable and pleasing to the consumer. The subject matter disclosed herein meets these and other needs.

### SUMMARY

In accordance with the purposes of the disclosed materials, compounds, compositions, articles, and methods, as embodied and broadly described herein, the disclosed subject matter, in one aspect, relates to compositions and methods for preparing and using such compositions. In a further aspect, the disclosed subject matter relates to methods of preparing salts of fatty acids (e.g., omega-3 fatty acids). In a still further aspect, the disclosed subject matter relates to compositions prepared by the methods disclosed herein. Also, disclosed are methods of using the disclosed compositions.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying Figures, which are incorporated in and constitute a part of this specification, illustrate several aspects described below.

FIG. 1 is a graph showing the concentration of omega-3 fatty acids in serum after supplementation with two preparations of omega-3 fatty acids as described in Example 8.

FIG. 2 is a graph showing the concentration of omega-3 fatty acids in red blood cells (RBCs) after supplementation with two preparations of omega-3 fatty acids as described in Example 8.

FIG. 3 is a graph showing the concentration of EPA and DHA in fecal samples after supplementation with two preparations of omega-3 fatty acids as described in Example 8.

### DETAILED DESCRIPTION

The materials, compounds, compositions, and methods described herein may be understood more readily by reference to the following detailed description of specific aspects of the disclosed subject matter and the Examples included therein and to the Figures.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### GENERAL DEFINITIONS

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a compound” includes mixtures of two or more such compounds, reference to “an acid” includes mixtures of two or more such acids, reference to “the salt” includes mixtures of two or more such salts, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data is provided in a number of different formats and that these data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

References in the specification and concluding claims to parts by weight of a particular component in a composition denotes the weight relationship between the component and any other components in the composition for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

By “treat” is meant to administer a composition disclosed herein (and/or a supplement, formulation, device, feed or foodstuff that contains the composition) to a subject or a sample in order to eliminate or reduce a disease or condition (e.g., diabetes or cardiovascular disease) within a subject or sample; stabilize or delay the progression of a disease or condition within a subject or sample; or decrease the frequency or severity of symptoms and/or recurrences of a disease or condition within a subject or sample.

By “prevent” is meant to minimize the chance that a subject will develop a disease or condition, or to delay the development of a disease or condition in a subject. For example, the compositions disclosed herein can be administered to minimize or delay the chance that a subject will develop diabetes. For subjects belonging to families having hereditary predisposition to various diseases and conditions, such as cardiovascular disease, compositions disclosed herein can be administered prior to disease onset or upon diagnosis, thereby lessening the chance that the subject will develop the particular disease or condition, and/or delaying the onset of the disease or condition, relative to the time that onset would have occurred, had the compositions (and/or a supplement, formulation, device, feed or foodstuff that contains the composition) not been administered.

Reference will now be made in detail to specific aspects of the disclosed materials, compounds, compositions, articles, and methods, examples of which are illustrated in the accompanying Examples and in the Figures.

### Materials and Compositions

Disclosed herein are materials, compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a compound is disclosed and a number of modifications that can be made to a number of components or residues of the compound are discussed, each and every combination and permutation that are possible are specifically contemplated unless specifically indicated to the contrary. Thus, if a class of components A, B, and C are disclosed as well as a class of components D, E, and F and an example of a combination composition A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed.

Disclosed herein, in one aspect, are compositions that contain salts of fatty acids, e.g., omega-3 fatty acids. These salts can be, as disclosed herein, calcium, magnesium, sodium, potassium, or zinc salts, including mixtures thereof. The term “salt” as used herein refers to the acyloxyl group RCOO— and its associated counterion(s) (e.g., Ca, Mg, Na, K, or Zn). The term “salt” is not meant to imply any particular stoichiometric relationship between the acyloxyl group(s) and the counterion(s), which can vary depending on such factors as the amount of hydration, the type of counterion, the valance and size of the counterion, the presence of other compounds, and the like. Also, it is said herein that a salt or composition is “derived” from a fatty acid. By this it is meant that the disclosed salt or composition is prepared directly or indirectly from a composition containing a fatty acid or residue thereof or the neat fatty acid or residue. Such methods are disclosed herein and include, for example, situations where a fatty acid or a fatty acid ester is converted to its corresponding salt, or where one fatty acid salt is converted into another fatty acid salt.

Specific compositions disclosed herein are compositions that contain at least one calcium salt of an omega-3 fatty acid. In another example, disclosed herein are compositions that contain at least one magnesium salt of an omega-3 fatty acid. Still further, disclosed herein are compositions that contain at least one sodium salt of an omega-3 fatty acid. In another example, disclosed herein are compositions that contain at least one potassium salt of an omega-3 fatty acid. Also disclosed are compositions that contain at least one zinc salt of an omega-3 fatty acid.

All combinations of these salts are also disclosed. For example, disclosed herein are compositions that contain at least one calcium salt of an omega-3 fatty acid and at least one magnesium salt of an omega-3 fatty acid, at least one calcium salt of an omega-3 fatty acid and at least one sodium salt of an omega-3 fatty acid, at least one calcium salt of an omega-3 fatty acid and at least one potassium salt of an omega-3 fatty acid, at least one calcium salt of an omega-3 fatty acid and at least one zinc salt of an omega-3 fatty acid, at least one magnesium salt of an omega-3 fatty acid and at least one sodium salt of an omega-3 fatty acid, at least one magnesium salt of an omega-3 fatty acid and at least one potassium salt of an omega-3 fatty acid, at least one magnesium salt of an omega-3 fatty acid and at least one zinc salt of an omega-3 fatty acid, at least one sodium salt of an omega-3 fatty acid and at least one potassium salt of an omega-3 fatty acid, at least one sodium salt of an omega-3 fatty acid and at least one zinc salt of an omega-3 fatty acid, or at least one potassium salt of an omega-3 fatty acid and at least one zinc salt of an omega-3 fatty acid.

Also disclosed are compositions that contains 3, 4, 5 or more different salts of omega-3 fatty acids. For example, disclosed herein are compositions comprising at least two salts chosen from a calcium salt of an omega-3 fatty acid, a magnesium salt of an omega-3 fatty acid, a sodium salt of an omega-3 fatty acid, a potassium salt of an omega-3 fatty acid, and a zinc salt of an omega-3 fatty acid.

The disclosed compositions can also comprise various amounts of omega-3 fatty acid residues. The term “residue” as used herein refers to the moiety that is the resulting product of the specified chemical species in a particular reaction scheme or subsequent formulation or chemical product, regardless of whether the moiety is actually obtained from the specified chemical species. For example, an “omega-3 fatty acid residue” refers to the moiety which results when an omega-3 fatty acid participates in a particular reaction (e.g., the residue can be an fatty acyl group RCO— or acyloxyl group RCOO—, where R is the hydrocarbon chain of the omega-3 fatty acid). In this case, the omega-3 fatty acid residue is “derived” from the omega-3 fatty acid. It is understood that this moiety can be obtained by a reaction with a species other than the specified omega-3 fatty acid, for example, by a reaction with an omega-3 fatty acid chloride, ester, or anhydride. Thus, when a composition is said to have a particular fatty acid residue, the residue can have the formula RCO₂X, where R is the hydrocarbon chain and X can be a hydrogen (i.e., the residue is a free, protonated fatty acid), alkyl group (e.g., the residue is a fatty acid ester or tirglyceride), or cation (i.e., the residue is a fatty acid salt).

In many examples, the compositions disclosed herein are derived or prepared from marine oils. Marine oils, as used herein, refer to oils isolated from marine life, which contain a wide variety of fatty acids. One or more of these fatty acids can be converted to their corresponding salt by the methods disclosed herein. Examples of suitable marine oils can be oils that are isolated from fish, Mollusca such as squid, cuttle fish, and/or octopus, Crustacea such as krill, and marine mammals such as seals and whales. Other specific examples of suitable marine oils include, but are not limited to, Atlantic fish oils, Pacific fish oils, Mediterranean fish oils, light pressed fish oil, alkaline treated fish oil, heat treated fish oil, light and heavy brown fish oil, tuna oil, bonito oil, sea bass oil, halibut oil, spearfish oil, barracuda oil, cod oil, menhaden oil, sardine oil, pilchard oil, anchovy oil, capelin oil, Atlantic cod oil, Atlantic herring oil, Atlantic mackerel oil, Atlantic menhaden oil, salmonids oil, shark oil, squid oil, octopus oil, krill oil, seal oil, whale oil, and the like, including mixtures and combinations thereof. Any marine oil and combination of marine oil can be used in the disclosed methods to prepare the disclosed compositions.

In other examples, the disclosed compositions can contain salts of fatty acids that are isolated from vegetables and plants, animals, and edible oils. In a specific example, the disclosed compositions can be isolated from microbial oil. Further examples of suitable oils include esterified oils from such sources disclosed herein. Still farther examples include crude oils, semi-refined (also called alkaline refined), and refined oils from such sources disclosed herein. Still further, the disclosed compositions and methods can use oils comprising re-esterified triglycerides. Also, any combination of these oils can be used.

Omega-3 Fatty Acids

The disclosed compositions can comprise one or more salts of omega-3 fatty acids. An omega-3 fatty acid is an unsaturated fatty acid that contains as its terminus CH₃—CH₂—CH═CH—. Specific examples of omega-3 fatty acids that can be present in the disclosed compositions include, but are not limited to, linolenic acid (18:3ω3), octadecatetraenoic:acid (18:4ω3), eicosapentaenoic acid (20:5ω3) (EPA), docosahexaenoic acid (22:6ω3) (DHA), docosapentaenoic acid (22:6ω3) (DPA), including residues, salts, derivatives, and mixtures thereof.

In many examples disclosed herein, the salts of omega-3 fatty acids can be derived from an omega-3 fatty acid residue having the following formula:

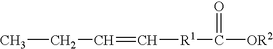

wherein R¹ is a C₃-C₄₀ alkyl or alkenyl group comprising at least one double bond and R² is H or alkyl group. The term “alkane” or “alkyl” as used herein is a saturated hydrocarbon group (e.g., methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like). The term “alkene” or “alkenyl” as used herein is a hydrocarbon group containing at least one carbon-carbon double bond. Asymmetric structures such as (AB)C═C(CD) are intended to include both the E and Z isomers (cis and trans). This may be presumed in structural formulae herein wherein an asymmetric alkene is present, or it may be explicitly indicated by the bond symbol C═C. In a further example, R¹ can be a C₅-C₃₈, C₆-C₃₆, C₈-C₃₄, C₁₀-C₃₂, C₁₂-C₃₀, C₁₄-C₂₈, C₁₆-C₂₆, or C₁₈-C₂₄ alkenyl group. In yet another example, the alkenyl group of R¹ can have from 2 to 6, from 3 to 6, from 4 to 6, or from 5 to 6 double bonds. Still further, the alkenyl group of R¹ can have from 1, 2, 3, 4, 5, or 6 double bonds, where any of the stated values can form an upper or lower endpoint as appropriate.

In some examples, the disclosed compositions can comprise at least about 10, 20, 30, 45, 60, or 75% of one or more omega-3 fatty acid residues by weight of the composition. In still other examples, the composition can comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of one or more omega-3 fatty acid residues by weight of the composition, where any of the stated values can form an upper or lower endpoint as appropriate. In further examples, the composition can comprise from about 10 to about 100, from about 20 to about 75, from about 30 to about 60, from about 10 to about 60, from about 20 to about 45, from about 30 to about 100, from about 45 to about 75, from about 60 to about 75, from about 45 to about 60, or from about 30 to about 45% of one or more omega-3 fatty acid residues by weight of the composition.

In some other examples, the disclosed compositions can contain less than about 10 weight % of conjugated linoleic acids.

Calcium, Magnesium, Sodium, Potassium, and/or Zinc Content

The disclosed compositions can contain various amounts of calcium, magnesium, sodium, potassium, and/or zinc. For example, the disclosed compositions can contain from about 1% to about 15% by weight of calcium, magnesium, sodium, potassium, zinc, or a combination thereof. In other examples, the disclosed compositions can contain from about 15 to about 1, from about 14 to about 1, from about 13 to about 1, from about 12 to about 1, from about 11 to about 1, from about 10 to about 1, from about 9 to about 1, from about 8 to about 1, from about 7 to about 1, from about 6 to about 1, from about 5 to about 1, from about 4 to about 1, from about 3 to about 1, from about 2 to about 1, from about 15 to about 2, from about 14 to about 2, from about 13 to about 2, from about 12 to about 2, from about 11 to about 2, from about 10 to about 2, from about 9 to about 2, from about 8 to about 2, from about 7 to about 2, from about 6 to about 2, from about 5 to about 2, from about 4 to about 2, from about 3 to about 2, from about 15 to about 3, from about 14 to about 3, from about 13 to about 3, from about 12 to about 3, from about 11 to about 3, from about 10 to about 3, from about 9 to about 3, from about 8 to about 3, from about 7 to about 3, from about 6 to about 3, from about 5 to about 3, from about 4 to about 3, from about 15 to about 4, from about 14 to about 4, from about 13 to about 4, from about 12 to about 4, from about 11 to about 4, from about 10 to about 4, from about 9 to about 4, from about 8 to about 4, from about 7 to about 4, from about 6 to about 4, from about 5 to about 4, from about 15 to about 5, from about 14 to about 5, from about 13 to about 5, from about 12 to about 5, from about 11 to about 5, from about 10 to about 5, from about 9 to about 5, from about 8 to about 5, from about 7 to about 5, from about 6 to about 5, from about 15 to about 6, from about 14 to about 6, from about 13 to about 6, from about 12 to about 6, from about 11 to about 6, from about 10 to about 6, from about 9 to about 6, from about 8 to about 6, from about 7 to about 6, from about 15 to about 7, from about 14 to about 7, from about 13 to about 7, from about 12 to about 7, from about 11 to about 7, from about 10 to about 7, from about 9 to about 7, from about 8 to about 7, from about 15 to about 8, from about 14 to about 8, from about 13 to about 8, from about 12 to about 8, from about 11 to about 8, from about 10 to about 8, from about 9 to about 8, from about 15 to about 9, from about 14 to about 9, from about 13 to about 9, from about 12 to about 9, from about 11 to about 9, from about 10 to about 9, from about 15 to about 10, from about 14 to about 10, from about 13 to about 10, from about 12 to about 10, from about 11 to about 10, from about 15 to about 11, from about 14 to about 11, from about 13 to about 11, from about 12 to about 11, from about 15 to about 12, from about 14 to about 12, from about 13 to about 12, from about 15 to about 13, from about 14 to about 13, or from about 15 to about 14% by weight of calcium, magnesium, sodium, potassium, zinc, or a combination thereof.

In other specific examples, when the composition comprises a calcium salt, the calcium content can be from about 6.0 to about 7.5 (e.g., 6.7%); when the composition comprises a magnesium salt, the magnesium content can be from about 4.0 to about 5.0 (e.g., 4.4%); when the composition comprises a potassium salt, the potassium content can be from about 11.0 to about 13.0 (e.g., 11.9%); when the composition comprises a sodium salt, the sodium content can be from about 6.0 to about 7.5 (e.g., 6.5%); and when the composition comprises a zinc salt, the zinc content can be from about 11.0 to about 13.0 (e.g., 11.7%).

Additional Fatty Acids

As noted, any oil can be used in the disclosed compositions and methods. Such oils can contain other fatty acids in addition to omega-3 fatty acids. Thus, compositions derived from such oils, as disclosed herein, can also comprise salts derived from these other fatty acids. It is also contemplated that while a particular fatty acid may not be present in the crude oil from which a specific composition is derived, such a fatty acid, residue, or salt derived therefrom can be added to the composition at any time (e.g., prior, during, or after the methods disclosed herein).

In some examples, the fatty acids, residues, and salts derived therefrom that can be present in the disclosed compositions can comprise at least 8, at least 10, at least 12, at least 14, at least 16, at least 18, or at least 20 carbon atoms. In some other examples, the fatty acids, residues, or salts derived therefrom can contain about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 carbon atoms, where any of the stated values can form an upper or lower endpoint as appropriate. In still other examples, the fatty acids, residues, and salts derived therefrom can comprise a mixture of fatty acids and salts having a range of carbon atoms. For example, the fatty acids, residues, and salts derived therefrom can comprise from about 8 to about 40, from about 10 to about 38, from about 12 to about 36, from about 14 to about 34, from about 16 to about 32, from about 18 to about 30, or from about 20 to about 28 carbon atoms.

The fatty acids, residues, and salts derived therefrom that can be present in the disclosed compositions can be saturated, unsaturated, or a mixture of saturated and unsaturated fatty acids or salts. By “saturated” is meant that the molecule or residue contains no carbon-carbon double or triple bounds. By “unsaturated” is meant that the molecule or residue contains at least one carbon-carbon double or triple bond. The disclosed compositions can also be processed to result in a particular mixture of fatty acids (e.g., having only saturated fatty acids, only unsaturated fatty acids, mixtures of both saturated and unsaturated fatty acids, or mixtures of fatty acids of a certain chain length or range of chain lengths).

### Saturated Fatty Acids, Residues, and Salts Derived Therefrom

Examples of saturated fatty acids, including residues and salts derived therefrom, that can be present in the disclosed compositions include, but are not limited to, the saturated fatty acids capric acid (C10), lauric acid (C12), myristic acid (C14), palmitic acid (C16), margaric acid (C17), stearic acid (C18), arachidic acid (C20), behenic acid (C22), lignoceric acid (C24), cerotic acid (C26), montanic acid (C28), and melissic acid (C30), including branched and substituted derivatives thereof.

It is contemplated that in many of the examples disclosed herein that amount of saturated fatty acids can be low, e.g., less than about 5, 4.5, 4.0, 3.5, 3.0, 2.5, 2.0, 1.5, or 1.0 wt. %.

### Unsaturated Fatty Acids, Residues, and Salts Derived Therefrom

Examples of unsaturated fatty acids, including residues and salts derived therefrom, that can be present in the disclosed compositions contain at least one unsaturated bond (i.e., a carbon-carbon double or triple bond). In one example, the unsaturated fatty acids, residues, and salts derived therefrom can comprise at least 2, at least 3, at least 4, at least 5, at least 6, at least 7, or at least 8 carbon-carbon double bonds, triple bonds, or any combination thereof. In another example, the unsaturated fatty acids, residues, or salts derived therefrom can comprise 1, 2, 3, 4, 5, 6, 7, or 8 unsaturated bonds, where any of the stated values can form an upper or lower endpoint as appropriate.

### Monoene Acids, Residues, and Salts Derived Therefrom

In one aspect, the unsaturated fatty acids, residues, or salts derived therefrom can comprise one carbon-carbon double bond (i.e., a monoene acid or residue). Examples of such unsaturated fatty acids, residues, and salts that can be present in the disclosed compositions include, but are not limited to, those in the following Table 1.

TABLE 1
Examples of Monoenes, Residues, and Salts Derived Therefrom

| Total number of | Carbon number where double bond begins. |
|---|---|
| carbon atoms in | (“c” denotes a cis double bond; “t” denotes a trans |
| the fatty acid chain | double bond) |
| 10 | 4c |
| 12 | 4c |
| 14 | 4c and 9c |
| 16 | 3t, 4c, 5t, 6c, 6t, 9c (palmitooleic), and 11c |
| 18 | 3t, 5c, 5t, 6c (petroselinic), 6t, 9c (oleic), 10c, |
| | 11c (cis-vaccenic), 11t (vaccenic), and 13c |
| 20 | 5c, 9c (gadolenic), 11c, 13c, and 15c |
| 22 | 5c, 11c (cetoleic), 13c (erucic), and 15c |
| 24 | 15c (selacholeic, nervonic) |
| 26 | 9c, and 17c (ximenic) |
| 28 | 9c, 19c (lumequic) |
| 30 | 21c |

### Polyene Acids, Residues and Salts Derived Therefrom

### Methylene Interrupted

In other examples, the unsaturated fatty acids, residues, and salts derived therefrom can comprise at least two unsaturated bonds (e.g., polyene acids or salts). In some examples, the unsaturated fatty acids, residues, and salts can comprise at least one pair of methylene interrupted unsaturated bonds. By “methylene interrupted unsaturated bond” is meant that one carbon-carbon double or triple bond is separated from another carbon-carbon double or triple bond by at least one methylene group (i.e., CH₂). Specific examples of unsaturated fatty acids, residues, and salts that contain at least one pair of methylene interrupted unsaturated bonds include, but are not limited to, the n−1 family derived from 9, 12, 15-16:3; n-2 family derived from 9, 12, 15-17:3, 15:3, 17:3, 17:4, 20:4; n-3 family derived from 9, 12, 15-18:3, 15:2, 15:3, 15:4, 16:3, 16:4, 18:3 (α-linolenic), 18:4, 18:5, 20:2, 20:3, 20:4; 20:5 (EPA), 21:5, 22:3, 22:5 (DPA), 22:6 (DHA), 24:3, 24:4, 24:5, 24:6, 26:5, 26:6, 28:7, 30:5; n-4 family derived from 9, 12-16:2, 16:2, 16:3, 18:2, 18:3; n-5 family derived from 9, 12-17:2, 15:2, 17:2, 17:3, 19:2, 19:4, 20:3, 20:4 21:4, 21:5; n-6 family derived from 9, 12-18:2, 15:2, 16:2, 18:2 (linoleic acid), 18:3 (Y-linolenic acid); 20:2, 20:3, 20:4 (arachidonic acid), 22:2, 22:3, 22:4 (adrenic acid), 22:5, 24:2, 24:4, 25:2, 26:2, 30:4; n-7 family derived from 9-16:1, 15:2, 16:2, 17:2, 18:2, 19:2; n-8 family derived from 9-17:1, 15:2, 16:2, 17:2, 18:2, 19:2; n-9 family derived from 9-18:1, 17:2, 18:2, 20:2, 20:3, 22:3, 22:4; n-11 family 19:2, and the n-12 family 20:2.

In the above paragraph (and throughout) the compounds are identified by referring first to the “n-x family,” where x is the position in the fatty acid where the first double bond begins. The numbering scheme begins at the terminal end of the fatty acid, where, for example, the terminal CH₃ group is designated position 1. In this sense, the n-3 family would be an omega-3 fatty acid, as described above. The next number identifies the total number of carbon atoms in the fatty acid. The third number, which is after the colon, designates the total number of double bonds in the fatty acid. So, for example, in the n-1 family, 16:3, refers to a 16 carbon long fatty acid with 3 double bonds, each separated by a methylene, wherein the first double bond begins at position 1, i.e., the terminal end of the fatty acid. In another example, in the n-6 family, 18:3, refers to an 18 carbon long fatty acid with 3 methylene separated double bonds beginning at position 6, i.e., the sixth carbon from the terminal end of the fatty acid, and so forth.

Some other examples are fatty acids, residues, and salts derived therefrom that contain at least one pair of unsaturated bonds interrupted by more than one methylene group. Suitable examples of these acids, residues, and salts include, but are not limited to, those in the following Table 2.

TABLE 2
Examples of Polyene Acids, Residues, and Salts Derived Therefrom
with Double Bonds Interrupted by Several Methylene Units

| Total number of | Carbon number where double bond begins. |
|---|---|
| carbon atoms | (“c” denotes a cis double bond; “t” denotes a |
| in the fatty acid chain | trans double bond) |
| 18 | 5, 9 |
| | 5, 11 |
| | 2t, 9, 12 |
| | 3t, 9, 12 |
| | 5t, 9, 12 |
| | 5, 9, 12 |
| | 5, 11, 14 |
| | 3t, 9, 12, 15 |
| | 5, 9, 12, 15 |
| 20 | 5, 11 |
| | 5, 13 |
| | 7, 11 |
| | 7, 13 |
| | 5, 11, 14 |
| | 7, 11, 14 |
| | 5, 11, 14, 17 |
| 22 | 5, 11 |
| | 5, 13 |
| | 7, 13 |
| | 7, 15 |
| | 7, 17 |
| | 9, 13 |
| | 9, 15 |

### Polyene Acids, Residues, and Salts Derived Therefrom

### Conjugated

Still other examples of unsaturated fatty acids, residues, and salts derived therefrom that can be present in the disclosed compositions are those that contain at least one conjugated unsaturated bond. By “conjugated unsaturated bond” is meant that at least one pair of carbon-carbon double and/or triple bonds are bonded together, without a methylene (CH₂) group between them (e.g., —CH═CH—CH═CH—). Specific examples of unsaturated fatty acids that contain conjugated unsaturated bonds include, but are not limited to, those in the following Table 3.

TABLE 3
Examples of Conjugated Polyene Acids, Residues, and Salts
Derived Therefrom

| Total number of | Carbon number where double bond begins. |
|---|---|
| carbon atoms | (“c” denotes a cis double bond; “t” denotes a |
| in the fatty acid chain | trans double bond) |
| 10 | 2t, 4t, 6c |
| | 2c, 4t, 6t |
| | 3t, 5t, 7c |
| | 3c, 5t, 7t |
| 12 | 3, 5, 7, 9, 11 |
| 14 | 3, 5, 7, 9, 11 |
| 18 | 10t, 12t |
| | 8c, 10t, 12c (jacaric) |
| | 8t, 10t, 12c (calendic) |
| | 8t, 10t, 12t |
| | 9t, 11t, 13c (catalpic) |
| | 9c, 11t, 13t (α-eleostearic) |
| | 9c, 11t, 13c (punicic) |
| | 9t, 11t, 13t (β-eleostearic) |
| | 9c, 11t, 13t, 15c (α-parinaric) |
| | 9t, 11t, 13t, 15t (β-parinaric) |

### Exemplary Unsaturated Fatty Acids, Residues, and Salts Derived Therefrom

Some specific examples of unsaturated fatty acids, residues, and salts derived therefrom that can be present in the disclosed compositions include, but are not limited to, linoleic acid, linolenic acid, γ-linolenic acid, arachidonic acid, mead acid, stearidonic acid, α-eleostearic acid, eleostearic acid, pinolenic acid, docosadienic acid, docosatetraenoic acid, docosapentaenoic acid, docosahexaenoic acid, octadecadienoic acid, octadecatrienoic acid, eicosatetraenoic acid, eicosapentaenoic, or any combination thereof. In one aspect, the unsaturated fatty acid, residue, or salt can be derived from eicosapentaenoic acid 20:5ω3 (EPA), docosahexaenoic acid 22:6ω3 (DHA), docosapentaenoic acid 22:5ω3 (DPA), and any combination thereof.

Additional examples of unsaturated fatty acids, residues, and salts derived therefrom that can be present in the disclosed compositions include, but are not limited to, allenic and acetylenic acids, such as, C14: 2, 4, 5; C18: 5, 6 (laballenic); 5, 6, 16 (lamenallenic); C18: 6a (tarinic); 9a; 9a, 11t (ximenynic); 9a, 11a; 9a, 11a, 13c (bolekic); 9a, 11a, 13a, 15e, 8a, 10t (pyrulic); 9c, 12a (crepenynic); 9c, 12a, 14c (dehydrocrepenynic acid); 6a, 9c, 12c; 6a, 9c, 12c, 15c, 8a, 11c, 14c and corresponding Δ17e derivatives, 8-OH derivatives, and Δ17e, 8-OH derivatives. Branched-chain acids, particularly iso-acids and anteiso acids, polymethyl branched acids, phytol based acids (e.g., phytanic, pristanic), furanoid acids are also suitable fatty acids, including the residues and salts derived therefrom, which can be present in the disclosed compositions. Still further fatty acids, residues, and salts derived therefrom include, but are not limited to, cyclic acids, such as cyclopropane fatty acids, cyclopropene acids (e.g., lactobacillic), sterulic, malvalic, sterculynic, 2-hydroxysterculic, aleprolic, alepramic, aleprestic, aleprylic alepric, hydnocarpic, chaulmoogric hormelic, manaoic, gorlic, oncobic, cyclopentenyl acids, and cyclohexylalkanoic acids. Hydroxy acids, particularly butolic, ricinoleic, isoricinoleic, densipolic, lesquerolic, and auriolic are also suitable fatty acids that can be present in the disclosed compositions. Epoxy acids, particularly epoxidated C18:1 and C18:2, and paranoid acids are further examples of fatty acids, residues, and salts derived therefrom that can be present in the disclosed compositions.

Amounts of DHA/EPA

As noted, many of the disclosed compositions can contain residues of the omega-3 fatty acids EPA and DHA. Each of these residues (e.g., in the form of either salts, such as calcium, magnesium, sodium, potassium, or zinc salts, esters, such as methyl, ethyl, or triglyceride esters, or free acids), can be present in the disclosed compositions in an amount of from about 0 to about 700 milligrams per gram of the composition. In other examples, the residues of DHA and/or EPA can each be present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, or 700 milligrams per gram of the composition, where any of the stated values can form an upper or lower endpoint as appropriate.

In still other examples, residues of DHA and/or EPA can each be present in the disclosed compositions in an amount from about 50 to about 700, from about 100 to about 700, from about 150 to about 700, from about 200 to about 700, from about 250 to about 700, from about 300 to about 700, from about 350 to about 700, from about 400 to about 700, from about 450 to about 700, from about 500 to about 700, from about 550 to about 700, from about 600 to about 700, from about 650 to about 700, from about 0 to about 650, from about 50 to about 650, from about 100 to about 650, from about 150 to about 650, from about 200 to about 650, from about 250 to about 650, from about 300 to about 650, from about 350 to about 650, from about 400 to about 650, from about 450 to about 650, from about 500 to about 650, from about 550 to about 650, from about 600 to about 650, from about 0 to about 600, from about 50 to about 600, from about 100 to about 600, from about 150 to about 600, from about 200 to about 600, from about 250 to about 600, from about 300 to about 600, from about 350 to about 600, from about 400 to about 600, from about 450 to about 600, from about 500 to about 600, from about 550 to about 600, from about 0 to about 550, from about 50 to about 550, from about 100 to about 550, from about 150 to about 550, from about 200 to about 550, from about 250 to about 550, from about 300 to about 550, from about 350 to about 550, from about 400 to about 550, from about 450 to about 550, from about 500 to about 550, from about 0 to about 500, from about 50 to about 500, from about 100 to about 500, from about 150 to about 500, from about 200 to about 500, from about 250 to about 500, from about 300 to about 500, from about 350 to about 500, from about 400 to about 500, from about 450 to about 500, from about 0 to about 450, from about 50 to about 450, from about 100 to about 450, from about 150 to about 450, from about 200 to about 450, from about 250 to about 450, from about 300 to about 450, from about 350 to about 450, from about 400 to about 450, from about 0 to about 400, from about 50 to about 400, from about 100 to about 400, from about 150 to about 400, from about 200 to about 400, from about 250 to about 400, from about 300 to about 400, from about 350 to about 400, from about 0 to about 350, from about 50 to about 350, from about 100 to about 350, from about 150 to about 350, from about 200 to about 350, from about 250 to about 350, from about 300 to about 350, from about 0 to about 300, from about 50 to about 300, from about 100 to about 300, from about 150 to about 300, from about 200 to about 300, from about 250 to about 300, from about 0 to about 250, from about 50 to about 250, from about 100 to about 250, from about 150 to about 250, from about 200 to about 250, from about 0 to about 200, from about 50 to about 200, from about 100 to about 200, from about 150 to about 200, from about 0 to about 150, from about 50 to about 150, from about 100 to about 150, from about 0 to about 100, from about 50 to about 100, from about 0 to about 50 milligrams per gram of composition.

The amount of EPA and DHA residues that can be present in the disclosed compositions can also be described in terms of weight % (wt. %). For example, the disclosed compositions can comprise from about 0 to about 70 wt. % EPA and/or DHA residues, based on the total weight of the composition. In other examples, the disclosed compositions can comprise about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 wt. % EPA and/or DHA residues based on the total weight of the composition, where any of the stated values can form an upper or lower endpoint as appropriate.

In still further examples, the amount of EPA and/or DHA residues that can be present in the disclosed composition can be from about 5 to about 70, from about 10 to about 70, from about 15 to about 70, from about 20 to about 70, from about 25 to about 70, from about 30 to about 70, from about 35 to about 70, from about 40 to about 70, from about 45 to about 70, from about 50 to about 70, from about 55 to about 70, from about 60 to about 70, from about 65 to about 70, from about 0 to about 65, from about 5 to about 65, from about 10 to about 65, from about 15 to about 65, from about 20 to about 65, from about 25 to about 65, from about 30 to about 65, from about 35 to about 65, from about 40 to about 65, from about 45 to about 65, from about 50 to about 65, from about 55 to about 65, from about 60 to about 65, from about 0 to about 60, from about 5 to about 60, from about 10 to about 60, from about 15 to about 60, from about 20 to about 60, from about 25 to about 60, from about 30 to about 60, from about 35 to about 60, from about 40 to about 60, from about 45 to about 60, from about 50 to about 60, from about 55 to about 60, from about 0 to about 55, from about 5 to about 55, from about 10 to about 55, from about 15 to about 55, from about 20 to about 55, from about 25 to about 55, from about 30 to about 55, from about 35 to about 55, from about 40 to about 55, from about 45 to about 55, from about 50 to about 55, from about 0 to about 50, from about 5 to about 50, from about 10 to about 50, from about 15 to about 50, from about 20 to about 50, from about 25 to about 50, from about 30 to about 50, from about 35 to about 50, from about 40 to about 50, from about 45 to about 50, from about 0 to about 45, from about 5 to about 45, from about 10 to about 45, from about 15 to about 45, from about 20 to about 45, from about 25 to about 45, from about 30 to about 45, from about 35 to about 45, from about 40 to about 45, from about 0 to about 40, from about 5 to about 40, from about 10 to about 40, from about 15 to about 40, from about 20 to about 40, from about 25 to about 40, from about 30 to about 40, from about 35 to about 40, from about 0 to about 35, from about 5 to about 35, from about 10 to about 35, from about 15 to about 35, from about 20 to about 35, from about 25 to about 35, from about 30 to about 35, from about 0 to about 30, from about 5 to about 30, from about 10 to about 30, from about 15 to about 30, from about 20 to about 30, from about 25 to about 30, from about 0 to about 25, from about 5 to about 25, from about 10 to about 25, from about 15 to about 25, from about 20 to about 25, from about 0 to about 20, from about 5 to about 20, from about 10 to about 20, from about 15 to about 20, from about 0 to about 15, from about 5 to about 15, from about 10 to about 15, from about 0 to about 10, from about 5 to about 10, from about 0 to about 5 wt. % based on the total weight of the composition. In some other specific examples, the amount of EPA and/or DHA residues that can be present in the disclosed compositions can be about 0.3, 5, 12, 18, 25, or 60 wt. % based on the total weight of the composition, where any of the stated values can form an upper or lower endpoint as appropriate.

The amount of EPA and DHA residues present in the disclosed compositions can also be described in terms of the wt. % ratio of EPA to DHA residue. For example, the wt. % ratio of EPA to DHA residue in the disclosed compositions can be about 40:20 (i.e., about 40 wt. % EPA residue to about 20 wt. % DHA residue, based on the total weight of the composition). Other wt. % ratios of EPA to DHA residue that can be present in the disclosed compositions include, but are not limited to, about 18:12, about 5:25, about 60:0.3, and about 0.8:60. Further wt. % ratios of EPA to DHA residue for the disclosed compositions can be about 0:70, 5:70, 10:70, 15:70, 20:70, 25:70, 30:70, 70:30, 70:25, 70:20, 70:15, 70:10, 70:5, 70:0, 0:65, 5:65, 10:65, 15:65, 20:65, 25:65, 30:65, 35:65, 65:35, 65:30, 65:25, 65:20, 65:15, 65:10, 65:5, 65:0, 0:60, 5:60, 10:60, 15:60, 20:60, 25:60, 30:60, 35:60, 40:60, 60:40, 60:35, 60:30, 60:25, 60:20, 60:15, 60:10, 60:5, 60:0, 0:55, 5:55, 10:55, 15:55, 20:55, 25:55, 30:55, 35:55, 40:55, 45:55, 55:45, 55:40, 55:35, 55:30, 55:25, 55:20, 55:15, 55:10, 55:5, 55:0, 0:50, 5:50, 10:50, 15:50, 20:50, 25:50, 30:50, 35:50, 40:50, 45:50, 50:50, 50:45, 50:40, 50:35, 50:30, 50:25, 50:20, 50:15, 50:10, 50:5, 50:0, 0:45, 5:45, 10:45, 15:45, 20:45, 25:45, 30:45, 35:45, 40:45, 45:45, 45:40, 45:35, 45:30, 45:25, 45:20, 45:15, 45:10, 45:5, 45:0, 0:40, 5:40, 10:40, 15:40, 20:40, 25:40, 30:40, 35:40, 40:40, 40:35, 40:30, 40:25, 40:20, 40:15, 40:10, 40:5, 40:0, 0:35, 5:35, 10:35, 15:35, 20:35, 25:35, 30:35, 35:35, 35:30, 35:25, 35:20, 35:15, 35:10, 35:5, 35:0, 0:30, 5:30, 10:30, 15:30, 20:30, 25:30, 30:30, 30:25, 30:20, 30:15, 30:10, 30:5, 30:0, 0:25, 5:25, 10:25, 15:25, 20:25, 25:25, 25:20, 25:15, 25:10, 25:5, 25:0, 0:20, 5:20, 10:20, 15:20, 20:20, 20:15, 20:10, 20:5, 20:0, 0:15, 5:15, 10:15, 15:15, 15:10, 15:5, 15:0, 0:10, 5:10, 10:10, 10:5, 10:0, 0:5, 5:5, or 5:0. In yet other examples, the disclosed compositions can comprise from about 14 to about 20 weight % of EPA residue and/or from about 10 to about 16 weight % of DHA residue. Another ratio of EPA to DHA that can be used in the disclosed compositions is about 290 mg/g EPA to about 235 mg/g DHA.

Trans Fatty Acids

In many of the compositions disclosed herein, the amount of trans-fatty acids can be low. For example, the amount of trans fatty acids can be less than about 40 wt. % trans fatty acids. Other ranges include, less than about 35, 30, 25, 20, 15, 10, and 5 wt. % trans fatty acid. Keeping the amount of trans fatty acids low in the disclosed compositions can result from choosing starting materials that are low in trans fatty acids. For example, vegetables such as palm oil, soybean oil, safflower oil, and the like can have greater than about 50 wt. % trans fatty acids. Thus, in one example, the disclosed compositions are not derived from, and the disclosed methods do not use, oils that contain greater that about 50, 45, 40, 35, or 30 wt. % trans fatty acids.

It is contemplated, however, that oils high in trans fatty acids can be used, or that trans fatty acids can be added, in the disclosed methods to produce compositions that are useful for ruminants.

### Methods of Making

The disclosed compositions can be prepared by methods disclosed herein. For example, compositions that contain calcium salts of fatty acids can be prepared using several starting sources of calcium. In one method, a starting composition comprising fatty acids in their natural triglyceride form can be hydrolyzed by hydrated CaO. In another method, fatty acids in the form of ethyl esters can be hydrolyzed by hydrated CaO. In yet another method, sodium or potassium salts of hydrolyzed fatty acids can be subjected to cation exchange by effect of hydrated CaO.

In a further method, a starting composition comprising fatty acids in their natural triglyceride form can be contacted with hydrated CaCl₂ or Ca(AcO)₂. In another method, fatty acids in the form of ethyl esters can be contacted with CaCl₂ or Ca(AcO)₂. Still further, sodium or potassium salts of hydrolyzed fatty acids can be subjected to cation exchange by effect of hydrated CaCl₂ or Ca(AcO)₂. This later method can also be achieved with other calcium salts, including, for example, calcium nitrate, fumarate, lactate, tri-calcium citrate, etc.

Compositions that contain magnesium salts of fatty acids can be prepared using several starting sources of magnesium. For example, sodium or potassium salts of fatty acids can be subjected to cation exchange by effect of hydrated MgCl₂ or Mg(AcO)₂. This can also be achieved by other soluble magnesium salts, including, for example, magnesium sulfate, bisulfite, nitrate, etc. Further, fatty acids in the form of ethyl esters or triglycerides can be contacted with, e.g., hydrated MgCl₂.

Compositions that contain zinc salts of fatty acids can be prepared using several starting sources of zinc. For example, sodium or potassium salts of fatty acids can be subjected to cation exchange by effect of hydrated ZnCl₂ or Zn(AcO)₂. This can also be achieved by other soluble zinc salts, including, for example, zinc sulfate, bisulfite, nitrate, etc. Further, fatty acids in the form of ethyl esters or triglycerides can be contacted with, e.g., hydrated ZnCl₂.

Compositions that contain sodium or potassium salts of fatty acids can be prepared using sodium or potassium hydroxide, respectively. For example, fatty acids in the form of ethyl esters or triglycerides can be contacted with sodium or potassium hydroxide.

In a particular example, disclosed herein is a method for preparing a composition comprising contacting a composition comprising an omega-3 fatty acid or derivative thereof with an alkaline earth metal chloride or acetate. Examples of suitable alkaline earth metal chlorides or acetates include calcium chloride, magnesium chloride, and magnesium acetate. With these reagents, the disclosed compositions can contain chloride ions and/or acetate ions.

In another example, disclosed herein is a method for preparing a composition comprising contacting a composition comprising an omega-3 fatty acid or derivative thereof with an alkaline metal hydroxide. Examples of suitable alkaline metal hydroxides include sodium hydroxide or potassium hydroxide.

In a further example, disclosed herein is a method for preparing a composition comprising contacting a composition comprising an omega-3 fatty acid or derivative thereof with zinc chloride. With this reagent, the disclosed compositions can contain chloride ions.

In the disclosed methods, compositions that comprise any of the omega-3 fatty acids or residues thereof disclosed herein can be used. For example, the compositions can be derived from microbial oils or marine oils as disclosed above, which contain omega-3 fatty acids. Moreover, derivatives of omega-3 fatty acids can also be used. By “derivatives” is meant the esters of the fatty acids (e.g., methyl and ethyl esters), salts of the fatty acids (e.g., sodium and potassium salts), and triglycerides, diglycerides, and monoglyceride derivatives. In certain specific examples, the omega-3 fatty acids used in the disclosed methods are not glycerides. Further, any of the additional fatty acids disclosed herein can be present in the compositions, including derivatives thereof.

The disclosed methods can be conducted under an inert atmosphere, e.g., under N₂ or argon. In other examples, any of the disclosed methods can be conducted under an ambient atmosphere (e.g., wherein the reaction is not conducted under a low oxygen atmosphere (e.g., where the oxygen level of the reaction is reduced by purging with an inert gas or vacuum).

Mixing

The composition comprising an omega-3 fatty acid or derivative thereof and alkaline earth metal chloride or acetate, zinc salt (e.g., ZnCl₂), or alkaline metal hydroxide can be mixed by any methods known in the art. “Mixing” is not meant to imply a particular outcome of mixing, such as the dissolution of any components to a particular level or the formation of a particular composition, such as homogeneous mixture, although such mixtures can be produced and some components can be dissolved by mixing. It can be desired that the mixing be vigorous. Mixing can be performed manually or by a mechanical mixing device such as, but not limited to, a static mixer, a magnetic stirrer, a shaker, spinner, or rotating device. Mixing can also be performed by forcing or bubbling a gas through the mixture or by sonication.

Mixing the composition comprising an omega-3 fatty acid or derivative thereof and alkaline earth metal chloride or acetate, zinc salt (e.g., ZnCl₂), or alkaline metal hydroxide can be performed for at least 1 minute. Mixing can also be performed for at least 1, 5, 15, 20, 25, 30, 35, 40, 45, 50, 55, 60, 65, 70, 75, 80, 85, 90, 95, or 100 minutes, where any of the stated values can form an upper or lower endpoint as appropriate.

Temperature

Contacting the composition comprising an omega-3 fatty acid or derivative thereof and alkaline earth metal chloride or acetate, zinc salt (e.g., ZnCl₂), or alkaline metal hydroxide can be performed at various temperatures, but, typically, the method can take place at an elevated temperature. The precise elevated temperature can depend on the particular starting composition and amount thereof being used, the particular alkaline earth metal chloride or acetate and the amount thereof being used, the particular pressure, preference, and the like. Suitable temperatures at which the disclosed methods can be performed include, but are not limited to, from about 20 to about 210° C., from about 30 to about 190° C., from about 40 to about 180° C., from about 50 to about 170° C., from about 60 to about 160° C., from about 70 to about 150° C., from about 80 to about 140° C., from about 90 to about 130° C., or from about 100 to about 120° C. In other examples, the composition and alkaline earth metal chloride or acetate can be heated to about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, or 210° C., where any of the stated values can form an upper or lower endpoint as appropriate.

It is also contemplated that the composition comprising the omega-3 fatty acid or derivate thereof can be heated prior to contacting with the alkaline earth metal chloride or acetate. Such a pre-heating step can be performed at any of temperatures and temperature ranges described herein.

Heating and/or pre-heating the composition can take place over a period of time, for example for at least 1, 10, 20, 30, 40, 50, 60, 70, 80, or 90 minutes. In some examples, the heating step is performed for from about 10 to about 20, from about 20 to about 30, from about 10 to about 30, from about 30 to about 60, from about 60 to about 90, from about 10 to about 90, or from about 30 to about 90 minutes. Further, after heating, the mixture can be allowed to cool from about 30 to about 60 minutes.

Pressure

In the disclosed methods, contacting the composition comprising the omega-3 fatty acid or derivative thereof and the alkaline earth metal chloride or acetate, zinc salt (e.g., ZnCl₂), or alkaline metal hydroxide can be conducted under reduced pressure. A suitable pressure is less than or equal to about 1 Torr or less than or equal to about 0.1 Torr. In other examples, the contacting step can be conducted at a pressure of less than or equal to about 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01, where any of the stated values can form an upper and/or lower endpoint when appropriate.

In other examples, the partial pressure of oxygen in the atmosphere can be greater than about 100 Torr.

### Supplements

Also, disclosed herein are nutritional supplements comprising the compositions disclosed herein. A nutritional supplement is any compound or composition that can be administered to or taken by a subject to provide, supply, or increase a nutrient(s) (e.g., vitamin, mineral, essential trace element, amino acid, peptide, nucleic acid, oligonucleotide, lipid, cholesterol, steroid, carbohydrate, and the like). In one aspect, disclosed herein are nutritional supplements comprising any of the compositions disclosed herein. For example, a nutritional supplement can comprise a composition comprising one or more calcium, magnesium, sodium, potassium, and/or zinc salts of an omega-3 fatty acid.

The nutritional supplement can comprise any amount of the compositions disclosed herein, but will typically contain an amount determined to supply a subject with a desired dose of an oil or particular fatty acid (e.g., EPA and/or DHA). The exact amount of composition required in the nutritional supplement will vary from subject to subject, depending on the species, age, weight and general condition of the subject, the severity of any dietary deficiency being treated, the particular mode of administration, and the like. Thus, it is not possible to specify an exact amount for every nutritional supplement. However, an appropriate amount can be determined by one of ordinary skill in the art using only routine experimentation given the teachings herein.

The nutritional supplement can also comprise other nutrient(s) such as vitamins other trace elements, minerals, and the like. Further, the nutritional supplement can comprise other components such as preservatives, antimicrobials, anti-oxidants, chelating agents, thickeners, flavorings, diluents, emulsifiers, dispersing aids, or binders.

The nutritional supplements are generally taken orally and can be in any form suitable for oral administration. For example, a nutritional supplement can typically be in a tablet, gel-cap, capsule, liquid, sachets, or syrup form.

The nutritional supplements can be designed for humans or animals, based on the recommended dietary intake for a given individual. Such considerations are generally based on various factors such as species, age, and sex as described above, which are known or can be determined by one of skill in the art. In one example, the disclosed supplements can be used as a component of feed for animals such as, but not limited to, livestock (e.g., pigs, chickens, cows, goats, horses, and the like) and domestic pets (e.g., cats, dogs, birds, and the like).

### Pharmaceutical Formulations

Also, pharmaceutical formulations comprising the compositions are disclosed herein. A suitable pharmaceutical formulation can comprise any of the disclosed compositions with a pharmaceutically acceptable carrier. For example, a pharmaceutical formulation can comprise composition comprising one or more calcium, magnesium, sodium, potassium, and/or zinc salts of omega-3 fatty acids and a pharmaceutically acceptable carrier. The disclosed pharmaceutical formulations can be used therapeutically or prophylactically.

By “pharmaceutically acceptable” is meant a material that is not biologically or otherwise undesirable, i.e., the material may be administered to a subject without causing any undesirable biological effects or interacting in a deleterious manner with any of the other components of the pharmaceutical formulation in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Suitable carriers and their formulations are described in Remington: The Science and Practice of Pharmacy, 21^{st} ed., Lippincott Williams & Wilkins, Philadelphia, Pa., 2005, which is incorporated by reference herein for its teachings of carriers and pharmaceutical formulations. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution can be from about 5 to about 8 (e.g., from about 7 to about 7.5). Further carriers include sustained release preparations such as semipermeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents and the like in addition to the compounds disclosed herein. Pharmaceutical formulations can also include one or more additional active ingredients such as antimicrobial agents, anti-inflammatory agents, anesthetics, and the like.

The pharmaceutical formulation can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed compounds can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, marine oils, and injectable organic esters such as ethyl oleate. Aqueous carriers include water, alcoholic/aqueous solutions, and emulsions or suspensions, including saline and buffered media. Parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations for topical administration may include ointments, lotions, creams, gels, drops, suppositories, sprays, liquids and powders. Conventional pharmaceutical carriers, aqueous, powder or oily bases, thickeners and the like can be desirable.

Pharmaceutical formulations for oral administration include, but are not limited to, powders or granules, suspensions or solutions in water or non-aqueous media, capsules, sachets, or tablets. Thickeners, flavorings, diluents, emulsifiers, dispersing aids, or binders can be desirable.

Some of the formulations can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### Delivery Devices

Any of the compositions described herein can be incorporated into a delivery device. Examples of delivery devices include, but are not limited to, microspheres, nanospheres or nanoparticles, liposomes, noisome, nanoerythrosome, solid-liquid nanoparticles, lotions, creams, sprays, or emulsions. In some other specific examples, the disclosed compositions can be incorporated into gels, gel capsules, or tablets. Other delivery devices can include powders or powders coated with a polymer. Such devices can be given orally or, in the case of powders for example, sprinkled onto food or beverages. Other examples of delivery devices that are suitable for non-oral administration include pulmospheres. Examples of particular other delivery devices useful herein are described below.

The disclosed compounds can be incorporated into liposomes. As is known in the art, liposomes are generally derived from phospholipids or other lipid substances.

Liposomes are formed by mono- or multi-lamellar hydrated liquid crystals that are dispersed in an aqueous medium. Any non-toxic, physiologically acceptable and metabolizable lipid capable of forming liposomes can be used. The disclosed compositions in liposome form can contain, in addition to a compositions disclosed herein, stabilizers, preservatives, excipients, and the like. Examples of suitable lipids are the phospholipids and the phosphatidyl cholines (lecithins), both natural and synthetic. Methods of forming liposomes are known in the art. See, e.g., Prescott, Ed., Methods in Cell Biology, Volume XIV, Academic Press, New York, p. 33 et seq., 1976, which is hereby incorporated by reference herein for its teachings of liposomes and their preparation.

In other examples, the liposomes can be cationic liposomes (e.g., DOTMA, DOPE, DC cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see e.g., Brigham et al, Am. J. Resp. Cell. Mol. Biol. 1989, 1:95-100; Felgner et al., Proc. Natl. Acad. Sci. USA 1987, 84:7413-7; and U.S. Pat. No. 4,897,355, which are incorporated by reference herein for their teachings of liposomes. As one example, delivery can be via a liposome using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures, standard in the art. Liposomes where the diffusion of the compound or delivery of the compound from the liposome is designed for a specific rate or dosage can also be used.

As described herein, niosomes are delivery devices that can be used to deliver the disclosed compositions. Noisomes are multilamellar or unilamellar vesicles involving non-ionic surfactants. An aqueous solution of solute is enclosed by a bilayer resulting from the organization of surfactant macromolecules. Similar to liposomes, noisomes are used in targeted delivery of, for example, anticancer drugs, including methotrexate, doxorubicin, and immunoadjuvants. They are generally understood to be different from transferosomes, vesicles prepared from amphiphilic carbohydrate and amino group containing polymers, e.g., chitosan.

As described herein, nanoerythrosomes are delivery devices that can be used to deliver the disclosed compositions. Nanoerythrosomes are nano-vesicles made of red blood cells via dialysis through filters of defined pore size. These vesicles can be loaded with a diverse array of biologically active molecules, including proteins and the compositions disclosed herein. They generally serve as ideal carriers for antineoplastic agents like bleomycin, actinomycin D, but can be used for steroids, other lipids, etc.

Artificial red blood cells are further delivery devices that can be used to deliver the disclosed compositions. Artificial red blood cells can be generated by interfacial polymerization and complex emulsion methods. Generally, the “cell” wall is made of polyphtaloyl L-lysine polymer/polystyrene and the core is made of a hemoglobin solution from sheep hemolysate. Hemoglobin loaded microspheres typically have particle sizes of from about 1 to about 10 mm. Their size, flexibility, and oxygen carrying capacity is similar to red blood cells.

Solid-lipid nanoparticles are other delivery devices that can be used to deliver the disclosed compositions. Solid-lipid nanoparticles are nanoparticles that are dispersed in an aqueous surfactant solution. They are comprised of a solid hydrophobic core having a monolayer of a phospholipid coating and are usually prepared by high-pressure homogenization techniques. Immunomodulating complexes (ISCOMS) are examples of solid-lipid nanoparticles. They are cage-like 40 nm supramolecular assemblies comprising of phospholipid, cholesterol, and hydrophobic antigens and are used mostly as immunoadjuvants. For instance, ISCOMs are used to prolong blood-plasma levels of subcutaneously injected cyclosporine.

Microspheres and micro-capsules are yet other delivery devices that can be used to deliver the disclosed compositions. In contrast to liposomal delivery systems, microspheres and micro-capsules typically do not have an aqueous core but a solid polymer matrix or membrane. These delivery devices are obtained by controlled precipitation of polymers, chemical cross-linking of soluble polymers, and interfacial polymerization of two monomers or high-pressure homogenization techniques. The encapsulated compound is gradually released from the depot by erosion or diffusion from the particles. Successful formulations of short acting peptides, such as LHRH agonists like leuprorelin and triptoreline, have been developed. Poly(lactide co-glycolide) (PLGA) microspheres are currently used as monthly and three monthly dosage forms in the treatment of advanced prostrate cancer, endometriosis, and other hormone responsive conditions. Leuprolide, an LHRH superagonist, was incorporated into a variety of PLGA matrices using a solvent extraction/evaporation method. As noted, all of these delivery devices can be used with the disclosed compositions.

Pulmospheres are still other examples of delivery devices that can be used herein. Pulmospheres are hollow porous particles with a low density (less than about 0.1 g/mL). Pulmospheres typically have excellent re-dispersibility and are usually prepared by supercritical fluid condensation technology. Co-spray-drying with certain matrices, such as carbohydrates, human serumi albumin, etc., can improve the stability of proteins and peptides (e.g., insulin) and other biomolecules for pulmonary delivery. This type of delivery could be also accomplished with micro-emulsions and lipid emulsions, which are ultra fine, thin, transparent oil-in-water (o/w) emulsions formed spontaneously with no significant input of mechanical energy. In this technique, an emulsion can be prepared at a temperature, which must be higher than the phase inversion temperature of the system. At elevated temperature the emulsion is of water-in-oil (w/o) type and as it cools at the phase inversion temperature, this emulsion is inverted to become o/w. Due to their very small inner phase, they are extremely stable and used for sustained release of steroids and vaccines. Lipid emulsions comprise a neutral lipid core (i.e., triglycerides) stabilized by a monolayer of amphiphilic lipid (i.e., phospholipid) using surfactants like egg lecithin triglycerides and miglyol. They are suitable for passive and active targeting.

There are other oral delivery systems under investigation that are based on osmotic pressure modulation, pH modulation, swelling modulation, altered density and floating systems, mucoadhesiveness etc. These formulations and time-delayed formulations to deliver drugs in accordance with circadian rhythm of disease that are currently in use or investigation can be applied for delivery of the disclosed compositions.

### Foodstuffs

Also disclosed herein are foodstuffs comprising any of the disclosed compositions. By “foodstuff” is meant any article that can be consumed (e.g., eaten, drank, or ingested) by a subject. In one example, the compositions can be used as nutritional supplements that are added to a foodstuff. For example, the disclosed compositions can be added to food or beverages. In this sense, the disclosed compositions can be prepared in, for example, a powdered form and contained in articles such as sachets or shakers, which can be used to pour or sprinkle the disclosed compositions onto and into food and beverages.

In some examples, the foodstuff is a baked good, a pasta, a meat product, a frozen dairy product, a milk product, a cheese product, an egg product, a condiment, a soup mix, a snack food, a nut product, a plant protein product, a hard candy, a soft candy, a poultry product, a processed fruit juice, a granulated sugar (e.g., white or brown), a sauce, a gravy, a syrup, a nutritional bar, a beverage, a dry beverage powder, a jam or jelly, a fish product, or companion pet food. In other examples, the foodstuff is bread, tortillas, cereal, sausage, chicken, ice cream, yoghurt, milk, salad dressing, rice bran, fruit juice, a dry beverage powder, liquid beverage, rolls, cookies, crackers, fruit pies, or cakes.

Foodstuffs can also include animal feed products, such as semi-dry pet food and moist pet food (e.g., dog and cat food). Foodstuff can also include livestock feed, e.g., ruminant feed.

### Methods of Use

The disclosed compositions also have a wide variety of uses. For example, the disclosed compositions (including the nutritional supplements, pharmaceutical formulations, delivery devices, and foodstuffs) can be used as a source of fatty acids (e.g., omega-3 fatty acids), lowering triglycerides and influencing diabetes related biochemistry, to name but a few uses. For example, disclosed herein are methods of supplementing omega-3 fatty acids in a subject by administering an effective amount of a composition disclosed herein, wherein the composition comprises a calcium, magnesium, sodium, potassium, and/or zinc salt of an omega-3 fatty acid. Also disclosed herein are methods of supplementing omega-3 fatty acids in a subject by administering to the subject an effective amount of nutritional supplements, feeds, pharmaceutical formulations, delivery devices, or foodstuffs made from or with the disclosed compositions.

Hundreds of clinical studies, in addition to numerous in vitro and in vivo experiments, have confirmed the beneficial effects of omega-3 fatty acids in a variety of disease conditions. One notable benefit of omega-3 fatty acids has been in the area of cardiovascular disease, for example, the prevention of cardiac arrythmias associated with sudden cardiac death. The largest study conducted to date is the GISSI trial, which was conducted in several sites in Italy over a 3½ year period (GISSI-Prevenzione Investigators, “Dietary supplementation with n-3 polyunsaturated fatty acids and vitamin E after myocardial infarction: results of the GISSI-Prevenzione trial,” Lancet 354:447-455, 1999). Approximately 11,000 patients, who suffered a recent myocardial infarction, were randomized to receive 850 mg/day omega-3 fatty acids, vitamin E (300 mg/day), both treatments, or placebo. Treatment with the omega-3 supplements significantly reduced the rate of death, non-fatal myocardial infarction (MI), and stroke compared to the placebo subjects, while no effect was seen for Vitamin E. In the Diet and Reinfarction Trial (DART), 1,015 post-MI patients were randomized to two groups. Subjects of one group were advised to eat fatty fish twice weekly or to consume fish oil supplements. The second group (control subjects) was not given dietary advice. After a 2-year period, the treatment group had a 29% reduction in mortality caused by coronary heart disease (CHD) mortality (Burr et al., “Effects of changes in fat, fish and fibre intakes on death and myocardial reinfarction: diet and reinfarction trial (DART)”. Lancet 334:757-761, 1989). Dolecek, in her 1992 review of the epidemiological study Multiple Risk Factor Intervention Trial (MRFIT), reported evidence which showed that fatty acids derived from fish oils were significantly inversely associated with 10-year cardiovascular mortality (p<0.006) in 6,200 high-risk males (Dolecek, “Epidemiological evidence of relationships between dietary polyunsaturated fatty acids and mortality in the Multiple Risk Factor Intervention Trial,” PSEBM 200:177-182, 1992). Thus, over a 10-year period, those consuming higher amounts of omega-3 fish oil had greater protection from heart disease as well as all cause mortality (p<0.02), compared to those consuming lesser amounts.

The effects of omega-3 fatty acids on atherosclerosis were studied by Von Schacky and colleagues in 1999, using a randomized, double blind, placebo-controlled trial (Von Schacky et al., “The effect of dietary omega-3 fatty acids on coronary atherosclerosis. A randomized, double-blind, placebo-controlled trial,” Annals of Internal Medicine 130:554-562, 1999). Two hundred patients with angiographically proven coronary artery disease received 3.4 g/day omega-3 for a 2-year period or placebo. Subjects receiving the omega-3 supplements showed less progression and more regression of coronary atherosclerosis versus placebo (p<0.041).

The triglyceride-lowering effects of omega-3 fatty acids have been documented in several clinical trials (Harris et al., “The reduction of post-prandial triglyceridemia in humans by dietary n-3 fatty acids,” J Lipid Res 29:1451-1460, 1988; Hwang et al., “Does vegetable oil attenuate the beneficial effects of fish oil in reducing risk factors for cardiovascular disease?” Am J Clin Nutr 66:89-96, 1997; Nordoy et al., “Individual effects of dietary saturated fatty acids and fish oil on plasma lipids and lipoproteins in normal men,” Am J Clin Nutr 57:634-639, 1993; and Adler et al, “Effect of garlic and fish oil supplementation on serum lipid and lipoprotein concentrations in hypercholesterolemic men,” Am J Clin Nutr 65:445-450, 1997). Given that post-prandial lipidemia occurs after the fat in high-fat diets has been absorbed, and that post-prandial lipids in the blood, such as triglycerides are known to be atherogenic, omega-3 fatty acids' ability to lower triglycerides demonstrates their antiatherogenic and anti-thrombotic potential. Elevated triglycerides in the blood, as well as an increased TG:HDL cholesterol ratio has been identified as a risk factor in cardiovascular disease (Gaziano et al., “Fasting triglycerides, high-density lipoprotein, and risk of myocardial infaction,” Circulation 96(8):2520-2525, 1997). This is particularly true in women, whose levels of triglyceride increase following menopause. The use of hormone replacement therapy (HRT) can also elevate triglycerides, further increasing risk. A group of researchers recently examined the effect of fish oil supplements on serum triglyceride concentrations in women receiving and not receiving HRT, using a double-blind, placebo-controlled trial (Stark et al., “Effect of a fish-oil concentrate on serum lipids in postmenopausal women receiving and not receiving hormone replacement therapy in a placebo-controlled, double-blind trial,” Am J Clin Nutr 72:389-94, 2000). A group of 36 post-menopausal women were randomly assigned to receive either 4.0 g EPA/DHA daily or placebo for 28 days. Supplementation with omega-3 fatty acids was associated with 26% lower serum triglyceride concentrations (p<0.0001), as well as 28% lower TG:HDL cholesterol ratios (p<0.01). The researchers concluded that this intervention resulted in 27% reduction in cardiovascular disease (CVD) risk in these post-menopausal subjects. This represents significantly greater cardiovascular benefit in reducing triglycerides for women compared to men (Austin et al., “Hypertriglyceridemia as a cardiovascular risk factor,” Am J Cardiology 81(4A):7B-12B, 1998). These researchers found that reductions of 10% in blood TG resulted in 17% reduction of CVD risk in women versus 7% CVD risk reduction in men.

The effects of omega-3 fatty acids on infant cognitive development and neurodevelopmental disorders, such as Attention Deficit Hyperactivity Disorder (ADHD) and dyslexia, as well as on other disorders such as depression, bipolar disorder, schizophrenia and Alzheimer's disease in the adult population, have been studied in recent years. Connor reviewed the importance of omega-3 fatty acids in fetal and infant brain and retinal development, noting that fetal and infant development represent the critical periods for acquisition of essential n-3 fatty acids (Connor, “importance of n-3 fatty acids in health and disease,” Am J Clin Nutr 71(suppl):171S-175S, 2000). During pregnancy, both maternal stores and dietary intake of these fatty acids determine the supply to the growing fetus. Providing fish oil or sardines to pregnant women leads to higher DHA concentrations in both maternal plasma, red blood cells, and in cord blood plasma at birth. Consistent with these findings, Van Houwelingen and colleagues also found evidence of improved DHA status in infants whose mothers consumed fish oil supplements during late pregnancy (Van Houwelingen et al., “Essential fatty acid status in neonates after fish-oil supplementation during late pregnancy,” British J Nutr 74:723-731, 1995).

It has also been found that term infants fed infant formula supplemented with DHA, to mirror average levels found in human milk from birth to 4 months, have significantly greater DHA concentrations in red blood cell phospholipids, which remain stable, compared to a reduction in levels in a standard formula placebo group. Supplementing the formula with DHA up to 4 months of age appears to be an efficient way to improve the DHA status of the study infants (Lapillonne et al., “Erythrocyte fatty acid composition in term infants fed human milk or a formula enriched with a low eicosapentaenoic acid fish oil for 4 months,” European J Pediatrics 159(1/2):49-53, 2000).

Of particular interest is whether an increase in DHA in blood plasma actually translates to developmental advantages for the infant. Birch and coworkers conducted a randomized controlled trial in which LCPUFA were added to standard formulas in term infants to measure the impact on mental development (Birch et al., “A randomized controlled trial of early dietary supply of long-chain polyunsaturated fatty acids and mental development in term infants,” Developmental Med Child Neurol 42:174-181, 2000). Fifty-six healthy term infants were randomized to receive standard formula or formula supplemented with DHA or DHA/AA (arachidonic acid) for a period of 17 weeks. Supplementation of formula with DHA/AA was associated with a significant mean increase in the Mental Development Index (MDI) of the Bayley Scales of Infant Development, 2^{nd} edition (BSID-II), compared to infants receiving the standard formula. The authors concluded that their data supported a long-term cognitive advantage of infant dietary DHA supply during the first 4 months of life.

Willatts and Forsyth reviewed the role of LCPUFA on infant cognition (“The role of long-chain polyunsaturated fatty acids in infant cognitive development,” Prostaglandins, Leukotrienes and Essential Fatty Acids 63:95-100, 2000). They pointed out that the inconsistent results reported with studies of LCPUFA and psychomotor development may be due to the global tests of development being insufficiently sensitive for detecting effects of LCPUFA on infant cognitive function. In contrast, studies assessing the influence of LCPUFA on development of specific cognitive behaviours have shown a significant advantage for supplemented infants.

Substantial amounts of research have been accumulated in the area of omega-3 fatty acids and their effects on attention deficit hyperactivity disorder (ADHD), bipolar disorder, dyslexia, depression, schizophrenia and Alzheimer's Disease (Stordy, “Docosahexaenoic acid: a dietary factor essential for individuals with dyslexia, attention deficit disorder and dyspraxia?” Chem Soc Rev 244:102-114, 1999; Stevens et al., “Essential fatty acid metabolism in boys with attention deficit hyperactivity disorder,” Am Clin Nutr 62:761-768, 1995; Stevens et al., “Omega-3 fatty acids in boys with behaviour, learning and health problems,” Physiol Behavior 59(4/5):915-920, 1996; Maes et al., “Lowered n-3 polyunsaturated fatty acids in serum phospholipids and cholesteryl esters of depressed patients,” Psychiatry Res 85:275-291, 1999; Stoll et al., “Omega-3 fatty acids in bipolar disorder: a preliminary double-blind, placebo-controlled trial,” Arch Gen Psych 56:407-412, 1999; Edwards et al., “Omega-3 polyunsaturated fatty acid levels in the diet and in red blood cell membranes of depressed patients,” J Affective Disorders 48:149-155, 1998; Conquer et al., “Fatty acid analysis of blood plasma of patients with Alzheimer's disease, other types of dementia, and cognitive impairment,” Lipids 35(12):1305-1312, 2000). Attention deficit hyperactivity disorder (ADHD) has been associated with reduced levels of essential fatty acids, including DHA, in the blood. Stevens and coworkers, in two studies, found lower concentrations of essential fatty acids, including omega-3 fatty acids, in ADHD subjects versus controls, as well as a positive relationship between reduced omega-3 fatty acid status and behaviour problems in children.

Stordy discovered that young adult dyslexics had impaired dark adaptation compared to non-dyslexic controls, and that supplementation with DHA resulted in improvements in dark adaptation after 1 month (Stordy, “Docosahexaenoic acid: a dietary factor essential for individuals with dyslexia, attention deficit disorder and dyspraxia?” Chem Soc Rev 244:102-114, 1999). The non-dyslexic control subjects did not experience any improvement following supplementation. Other experiments demonstrated that dyslexic adults and children exhibit significantly more severe fatty acid deficiency than controls (p<0.03). Richardson and colleagues also found that subjects with dyslexia are deficient in long chain polyunsaturated fatty acids compared to controls (Richardson et al., “Is developmental dyslexia a fatty acid deficiency syndrome?” Nutrition Society Summer Meeting, Guildford, UK 30th June-3rd July, 1998).

Reduction in omega-3 fatty acids in serum phospholipids has been reported in subjects suffering from depression (Maes et al., “Lowered n-3 polyunsaturated fatty acids in serum phospholipids and cholesteryl esters of depressed patients,” Psych Res 85:275-291, 1999). Researchers reported an abnormal metabolism of n-3 PUFAs in subjects with depression, and concluded that the reduced levels of omega-3 fatty acids observed were related to an inflammatory response associated with the disease. Stoll and colleagues randomly assigned 30 adults with bipolar disorder either 9.6 g omega-3 fatty acids or placebo daily for a 4-month period (Stoll et al., “Omega-3 fatty acids in bipolar disorder: a preliminary double-blind, placebo-controlled trial,” Arch Gen Psych 56:407-412, 1999). The treatment group had a significantly longer period of remission compared to the placebo (p=0.002), as well as a significantly better Clinical Global Impression Scale score versus the placebo at 4-months (p<0.001). They also scored better than the placebo group on both the Global Assessment Scale (p=0.03) and the Hamilton Rating Scale for Depression (p=0.002). The authors concluded that the omega-3 supplements were well tolerated and improved the short-term course of illness in these subjects.

Research conducted by Mellor and colleagues has demonstrated improvements in schizophrenic symptoms, general psychopathology, and increased levels of EPA and DHA in red blood cell (RBC) phospholipids, following supplementation with 10 g EPA+DHA daily for a 6- to 8-week period (Mellor et al., “Eicosapentaenoic acid and schizophrenia,” Neuropsychopharmacol 10(3S Part 2):256S, 1994; Mellor et al, “Schizophrenic symptoms and dietary intake of n-3 fatty acids,” Schizophrenia Res 18:85-86, 1995; Mellor et al., “Omega-3 fatty acid supplementation in schizophrenic patients,” Human Psychopharmacol 11:39-46, 1996). The effect of omega-3 fatty acids on the risk of developing Alzheimer's disease and dementia was studied using data from the Rotterdam study, an epidemiological study published in 1991 (Kalmijn et al., “Dietary fat intake and the risk of incident dementia in the Rotterdam Study,” Ann Neurol 42:776-782, 1997). A total of 1%, 58 of the 5,386 participants, developed dementia over the 2-year study period, and 42 of those were diagnosed with Alzheimer's disease. Fish consumption (even at relatively low levels) was related to a reduced risk of dementia, and, in particular, a reduced risk of Alzheimer's disease. High intakes of total and saturated fat as well as cholesterol increased the risk.

Conquer and colleagues compared the blood plasma of subjects with Alzheimer's disease and other types of dementia to those of normal subjects to determine if differences in DHA existed in the blood (Conquer et al., “Fatty acid analysis of blood plasma of patients with Alzheimer's disease, other types of dementia, and cognitive impairment,” Lipids 35(12):1305-1312, 2000). This question was raised because reduced levels of DHA have been found in the brains of Alzheimer's patients. They found decreased levels of DHA, total n-3 fatty acids and n-3/n-6 ratio in plasma phospholipid and phosphatidyl choline (PC) fractions of blood plasma in the Alzheimer's (AD), other dementia (OD) and cognitively impaired/not demented (CIND) groups compared to the normal subjects.

The effects of omega-3 fatty acids on inflammatory bowel diseases (IBD), including Crohn's disease (CD) and ulcerative colitis (UC) was studied by Belluzzi and coworkers (Belluzzi et al., “Effect of an enteric-coated fish-oil preparation on relapses in Crohn's disease,” New Engl J Med 334(24):1557-1560, 1996). An enteric-coated fish oil capsule was found to be effective in reducing the rate of relapse for adults with Crohn's disease who were in remission, not taking drug therapy and who were at high risk of recurrence. In this double-blind study, 78 subjects were randomly assigned either 2.7 g (1.8 g EPA/0.9 g DHA) enteric-coated omega-3 or placebo daily for one year. The group receiving fish oil had a 28% reduction in the rate of relapse compared to 69% for the placebo (p<0.001). At the end of the study, 59% of the fish oil group was still in remission, compared to only 26% of the placebo group (p<0.003). Given the long-term negative effects of traditional IBD drug therapies on organ and immune function, fish oil is a virtually side-effect-free and attractive adjunct to drug therapy in the treatment of these diseases.

Studies examining potential benefits of omega-3 fatty acid supplementation in subjects with rheumatoid arthritis (RA) have yielded mostly positive results. However, some of the earlier studies used very high doses of fish oil, which can result in compliance problems due to unwanted gastrointestinal side effects (Cleland et al, “Clinical and biochemical effects of dietary fish oil supplements in rheumatoid arthritis,” Arthritis Rheumatism 15:1471-1475, 1988; Sperling et al, “Effects of dietary supplementation with marine fish oil on leukocyte lipid mediator generation and function in rheumatoid arthritis,” Arthritis Rheumatism 30(9):988-997, 1987; Kremer et al., “Effects of high-dose fish oil on rheumatoid arthritis after stopping nonsteroidal antiinflammatory drugs. Clinical and immune correlates,” Arthritis Rheumatism 38(8):1107-1114, 1995). Fortin and coworkers conducted a meta-analysis of 10 research studies using fish oil in subjects with rheumatoid arthritis (Fortin et al., “Validation of a meta-analysis: the effects of fish oil in rheumatoid arthritis,” J Clin Epidemiol 48(11): 1379-1390, 1995). A review of the journal articles allowed them to conclude that the use of fish oil significantly reduced the number of tender joints (p<0.05) and duration of morning stiffness in subjects with rheumatoid arthritis at 3 months (0.05≦p≦0.08) when compared to placebo subjects. Two long-term studies found positive effects of fish oil supplementation on the course of RA (Geusens et al., “Long-term effect of omega-3 fatty acid supplementation in active rheumatoid arthritis. A 12-month, double-blind, controlled study,” Arthritis Rheumatism 37(6):824-829, 1994; Lau et al., “Effects of fish oil supplementation on non-steroidal anti-inflammatory drug requirement in patients with mild rheumatoid arthritis—a double-blind placebo controlled study,” British JRheumatol 32(11):982-989, 1993). Geusens' group conducted a 12-month double blind trial, where 90 subjects with active RA were randomly assigned 1.7 g EPA, 0.85 g EPA or olive oil placebo (6 g) daily. The group receiving 1.7 g EPA daily had significant improvement in global assessment from baseline and throughout the study (p<0.05). Their pain score improved significantly (p<0.05), and their grip strength improved significantly (p<0.05). These symptoms worsened in the placebo group (p<0.01). In a 1993 study Lau and colleagues randomly assigned 64 RA patients either 1.7 g EPA or air-filled placebo capsules daily for 12 months, followed by a 3-month placebo period for both groups (Lau et al., “Effects of fish oil supplementation on non-steroidal anti-inflammatory drug requirement in patients with mild rheumatoid arthritis—a double-blind placebo controlled study,” British J Rheumatol 32(11):982-989, 1993). The fish oil group experienced a decrease in NSAID usage by 59% compared to a 16% reduction for the placebo group, and this effect persisted at 15 months (p<0.001). The fish oil group, while decreasing their NSAID consumption, did not experience any deterioration in clinical and laboratory parameters of RA activity.

In other examples, the disclosed compositions can also be used as a source or calcium, magnesium, sodium, potassium, and/or zinc. Calcium is an important element for health; in fact, calcium is the most common mineral in the body. Calcium forms a salt with phosphate called hydroxyapatite that gives bones and teeth their structural rigidity. The movement of calcium ions into and out of cells plays a key regulatory role in a number of physiological systems. These include constriction/relaxation of blood vessels, which regulates blood pressure, muscle contraction including that of cardiac muscle, neuronal transmission, secretion of hormones (e.g., insulin) and the modulation of intermediary metabolism. In addition, calcium plays a co-factor role for the enzymes involved in blood coagulation. A well recognized heath benefit of calcium is its use to support bone heath and the prevention of osteoporosis. According to National Academy of Sciences, the recommended dose allowance for calcium is 800 mg a day, 1200 mg a day for young men and women and lactating women.

Magnesium is an essential mineral that also an important role in health. Magnesium is a cofactor for a large number of enzymes of intermediary metabolism, particularly those involving ATP or GTP. These include glycolytic, citric acid cycle, and beta-oxidation enzymes. Thus the metabolism of carbohydrate and fatty acids is critically dependent on magnesium. Likewise, anabolic processes such as fatty acid, protein, carbohydrate and DNA/RNA synthesis involve magnesium-dependent enzymes. In addition, magnesium ions help maintain normal muscle and nerve function, including heart contractility. Magnesium also helps regulate immune system function and bone health. For example, when the body does not get enough magnesium, secretion of parathyroid hormone is diminished, which regulates blood calcium level and formation of vitamin D in the kidneys. Deficiencies of magnesium have also been linked to cardiovascular risk factors. Magnesium as a supplement is viewed to have a number of general health benefits including maintenance of a health cardiovascular system and blood pressure, prevention of osteoporosis, relieve of fibromyalgia, reduction in muscle pain and cramping, and relief of premenstrual syndrome symptoms. Magnesium is also recommended for patients with type 2 diabetes mellitus.

Potassium is the major intracellular cation in the body. It plays a central role in the electrochemical gradient across cell membranes known as the membrane potential. Potassium concentrations are higher inside than outside cells, whereas sodium concentrations are lower inside than outside cells. This is maintained through the action of ion pumps, in particular the sodium-potassium ATPase, a membrane protein complex that pumps potassium in and sodium out of cells. The membrane potential is critical for nerve transmission, muscle contraction, heart function, and the transport of various substances (e.g., nutrients) in and out of cells. The most recognized health benefit of potassium is for maintaining healthy blood pressure.

Zinc is also an essential mineral. It functions as co-factor for a variety of enzymes including RNA polymerases, alkaline phosphatase, carbonic anhydrases, and superoxide dismutases. Zinc is a structural component, as in the case of Zinc-finger motifs in many proteins (e.g., the retinoic acid receptor). Among it various physiological effects include protection against lipid peroxidation, immune system support, mineralization of bone, detoxification of substances in the liver, support of DNA synthesis, and the conversion of calorie-containing nutrients to energy. Zinc supplements are taken for a variety of general health purposes including wound healing, slowing of macular degeneration, and immune function. For example, there are a number of cold/flu products that contain zinc.

Thus, by administering to a subject an effective amount of any of the compositions disclosed herein that contain calcium, magnesium, sodium, potassium, and/or zinc salts of omega-3 fatty acids, one can supplement calcium, magnesium, sodium, potassium, and/or zinc intake, obtaining the benefits associated with the omega-3 fatty acids and the metals calcium, magnesium, potassium, sodium, and/or zinc.

As noted, disclosed herein are methods of lowering cholesterol levels, triglyceride levels, or a combination thereof in a subject by administering an effective amount of a composition disclosed herein, including nutritional supplements, feeds, pharmaceutical formulations, delivery devices, or foodstuffs made therefrom or therewith, to the subject.

Still further, disclosed are methods of improving insulin sensitivity, reducing hyperglycemia, reducing hypercholesterolemia, and/or treating or preventing diabetes in a subject by administering to the subject a composition disclosed herein, including nutritional supplements, feeds, pharmaceutical formulations, delivery devices, or foodstuffs made therefrom or therewith.

In yet further examples, disclosed herein are methods of reducing body fat and/or promoting weight loss in a subject by administering to the subject an effective amount of a composition disclosed herein, including nutritional supplements, feeds, pharmaceutical formulations, delivery devices, or foodstuffs made therefrom or therewith.

Also disclosed herein are methods of lowering blood pressure in a subject by administering to the subject an effective amount of the a composition disclosed herein, including nutritional supplements, feeds, pharmaceutical formulations, delivery devices, or foodstuffs made therefrom or therewith. Further, disclosed herein are methods of modulating arrhythmia, thrombosis, and/or inflammation in a subject by administering to the subject an effective amount of the a composition disclosed herein, including nutritional supplements, feeds, pharmaceutical formulations, delivery devices, or foodstuffs made therefrom or therewith.

A method of treating or preventing depression in a subject by administering to the subject an effective amount of the a composition disclosed herein, including nutritional supplements, feeds, pharmaceutical formulations, delivery devices, or foodstuffs made therefrom or therewith is also disclosed.

Still further, disclosed herein are methods of modulating development in an infant (e.g., visual development and/or cognitive development) by administering an effective amount of a composition disclosed herein, including nutritional supplements, feeds, pharmaceutical formulations, delivery devices, or foodstuffs made therefrom or therewith to the infant.

Also, disclosed herein are methods of treating or preventing rheumatoid arthritis in a subject (e.g., visual development and/or cognitive development) by administering an effective amount of a composition disclosed herein, including nutritional supplements, feeds, pharmaceutical formulations, delivery devices, or foodstuffs made therefrom or therewith to the subject.

In the disclosed methods, the compositions can be any of the compositions disclosed herein. Also, the disclosed compositions can be used neat or in combination with some other component. For example, the disclosed compositions can be used in the disclosed methods in the form of any of the nutritional supplements disclosed herein. In another example, the disclosed compositions can be used in the disclosed methods in the form of any of the pharmaceutical formulations disclosed herein. In still another example, the disclosed compositions can be incorporated in any of the delivery devices disclosed herein, or incorporated into any foodstuff disclosed herein and used in the disclosed methods.

It is contemplated that the methods disclosed herein can be accomplished by administering various forms of the disclosed compositions. For example, one can administer any of the pharmaceutical formulations with any of the foodstuffs disclosed herein. In another example, one can administer a tablet or capsule with any of the nutritional supplements disclosed herein. In yet another example, one can administer any of the pharmaceutical formulations with any of the delivery devices and nutritional supplement disclosed herein, and the like.

Dosage

When used in the above described methods or other treatments, or in the nutritional supplements, pharmaceutical formulations, delivery devices, or foodstuffs disclosed herein, an “effective amount” of one of the disclosed compositions can be employed in pure form or, where such forms exist, in pharmaceutically acceptable salt form, and with or without a pharmaceutically acceptable excipient, carrier, or other additive.

The specific effective dose level for any particular subject will depend upon a variety of factors including the disorder being treated and the severity of the disorder; the identity and activity of the specific composition employed; the age, body weight, general health, sex and diet of the patient; the time of administration; the route of administration; the rate of excretion of the specific composition employed; the duration of the treatment; drugs used in combination or coincidental with the specific composition employed and like factors well known in the medical arts. For example, it is well within the skill of the art to start doses of the composition at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose.

The dosage can be adjusted by the individual physician or the subject in the event of any counter-indications. Dosage can vary, and can be administered in one or more dose administrations daily, for one or several days. Guidance can be found in the literature for appropriate dosages for given classes of pharmaceutical products.

Administration and Delivery

In one aspect, disclosed herein are uses of a delivery device to deliver a disclosed composition to a subject. Further, disclosed are methods for delivering a disclosed composition to a subject by administering to the subject any of the nutritional supplements, pharmaceutical formulations, delivery devices, and/or foodstuffs disclosed herein.

The disclosed compositions (including nutritional supplements, delivery devices, and pharmaceutical formulations) can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of conditions, e.g., component concentrations, temperatures, pressures, and other reaction ranges and conditions that can be used to optimize the product purity and yield obtained from the described process. Only reasonable and routine experimentation will be required to optimize such process conditions.

Certain materials, compounds, compositions, and components disclosed herein can be obtained commercially or readily synthesized using techniques generally known to those of skill in the art. For example, the starting materials and reagents used in preparing the disclosed compositions are either available from commercial suppliers such as Ocean Nutrition Canada, Ltd. (Dartmouth, Canada), Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

The starting oil used in Examples 1-7 was 40:20 EE oil from Ocean Nutrition Canada (Dartmouth, Canada), which contains about 40 wt. % EPA ethyl ester and about 20 wt. % DHA ethyl ester (e.g., EPA 414 mg/g; DHA 211 mg/g; total omega-3 content (which includes EPA, DHA, and other omega-3 fatty acids like DPA) 700 mg/g).

The method to determine the EPA and DHA composition (as well as total omega-3) of the Ca/Mg/Na/K/Zn-omega 3 salts was the saponification-methylation method. The salts were saponified with a sodium hydroxide in methanol solution at 100° C. for 7 minutes, followed by methylation with a boron trichloride-methanol solution at 100° C. for 30 minutes. The fatty acid methyl esters were extracted with iso-octane and a sodium chloride solution. Analysis of the fatty acid methyl esters was by gas chromatography.

Specifically, the EPA, DHA, and total omega-3 fatty acid content of the salt compositions prepared in Examples 1-7 were determined as follows. A test solution was first prepared by adding the salt composition and an internal standard (C_{23:0}; 70 mg) into a volumetric flask. The sample and internal standard were diluted to mark with a 0.05 g/L solution of butylhydroxytoluene in tetrahydrofuran (THF). A portion of the solution (about 2.0 mL) was then pipetted into a test tube and evaporated to dryness with nitrogen. Sodium hydroxide (20 g/L solution in methanol) was then added and the solution was mixed and heated at 100° C. for 7 minutes. After cooling, a boron trichloride-methanol solution (2 mL) was next added. This solution was then mixed and heated at 100° C. for 30 minutes. After cooling to 40-50° C.; iso-octane and saturated NaCl solution were added with thorough mixing.

The upper layer of the resulting biphasic system was collected, washed with water, and dried with anhydrous sodium sulfate. The resulting solution was placed into a test tube and blown down to dryness with nitrogen gas. Next the sample was brought to volume with iso-octane using a calibrated pipette.

A reference solution was prepared by placing of docosahexaenoic acid ethyl ester (60.0 mg), an internal standard C_{23:0} methyl ester (70 mg), and eicosapentaenoic acid ethyl ester (90.0 mg) into a volumetric flask. The solution was diluted to mark with 0.05 g/L butylhydroxytoluene in iso-octane. The sample was methylated as described above for the test solution.

All samples were transferred to a vial and 1 μL was injected into a Gas Chromatograph (Hewlett-Packard 6890). One of the three GC conditions shown in Table 4 were used.

TABLE 4
GC conditions for Analysis of EPA, DHA, and total omega-3 acid content

| | Restek | | |
|---|---|---|---|
| | 6890/Resteck | | |
| | Famewax | Supelco | |
| | (30 m × | Omegawax | |
| | 0.32 mm × | (30 m × 0.32 mm × | Either |
| Column | 0.25 μm) (10 min) | 0.25 μm) (16 min) | (45 min) |
| FID temp (° C.) | 275 | 275 | 275 |
| Injector temp (° C.) | 250 | 250 | 250 |
| Carrier flow (mL/min H₂) | 3 | 3.2 | 1 |
| Split ratio | 100:1 | 100:1 | 100:1 |
| Make-up flow (mL/min He) | 45 | 45 | 30 |
| Oven temp 1 (° C.) | 195 for 0 min | 180 for 0 min | 100 for 0 min |
| Oven ramp 1 (° C./min) | 5 | 8 | 8 |
| Oven temp 2 (° C.) | 240 for 1 min | 210 for 12 min | 210 for 30 min |

For the metal analysis, a portion of each sample was digested with nitric acid in a hotblock digestor for several hours and then diluted with deionized water. These solutions were analyzed by Inductively Coupled Plasma Mass Spectrometry and Inductively Coupled Plasma Emission Spectrometry for the various metals. A portion of the acid digestion was further digested with sulfuric acid and potassium permanganate. This solution was analyzed for Mercury by Cold Vapor Atomic Absorption Spectrometry.

Halide analysis was performed in one of two ways. For low level samples, a small portion of sample was analyzed for Total Halides by combustion/microcoulometric titration. The samples that contained high levels of chloride cannot be analyzed by this method so they were extracted in a dilute nitric acid solution and this solution was analyzed for chloride colourimetrically.

### Example 1

### Use of Calcium Oxide

A suspension of 40.0 g (118 mmol) of 40:20 EE and 4.37 g (78 mmol) of CaO was heated under stirring to reach 120° C. and then 15.6 mL of water was added dropwise. The reaction was refluxed at 120° C. for 5 h under nitrogen. After cooling, the aqueous liquid was decanted and the solid material was lyophilized and pulverized. The resulting beige powder was solubilized in THF and the insolubles were removed by centrifugation. Total omega-3 content: 612 mg/g (86% yield). EPA content: 363 mg/g (86% yield). DHA content: 180 mg/g (84% yield). Calcium content was 7.0%.

### Example 2

### Use of Calcium Chloride

A mixture of 40.0 g (118 mmol) of 40:20 EE, 7.3 g ethanol, 6 g (150 mmol) NaOH in 6 g of water was heated to 80-85° C. and the reaction refluxed for 1 h under nitrogen. Then 11.4 g (78 mmol) of CaCl₂.2H₂O dissolved in 15.3 g water was added and the reaction refluxed (100-105° C.) for 5 h. After cooling, the liquid was decanted and the solid material lyophilized and pulverized into a beige powder. The powder was washed several times with water and re-lyophilized. Total omega-3 content: 667 mg/g (93% yield). EPA content: 393 mg/g (93% yield). DHA content: 199 mg/g (92% yield). Calcium content was 6.7%.

### Example 3

### Use of Magnesium Chloride

The magnesium salt was prepared as described in Example 2 except that 15.8 g (78 mmol) MgCl₂.6H₂O in 21.2 g of water was used. The resulting salt was a beige powder. Total omega-3 content: 688 mg/g (94% yield). EPA content: 407 mg/g (94% yield). DHA content: 205 mg/g (93% yield). Magnesium content was 4.4%. Sodium content was 1.1%.

### Example 4

### Use of Magnesium Acetate

The magnesium salt was prepared as described in Example 2 except that (AcO)₂Mg.2H₂O (6.97 g, 32.5 mmol) dissolved in 5 mL of water was used. The resulting salt was a white powder. Yield 81%. Magnesium content was 2.72%. EPA content was 344 mg/g. DHA content was 171 mg/g. Total FFA content was 598 mg/g.

### Example 5

### Use of Zinc Chloride

The zinc salt was prepared as described in Example 2 except that 10.64 g (78 mmol) ZnCl₂ in 7.3 g of water was added. After lyophilization the zinc salt was dissolved in hexane, filtered and evaporated. The product was obtained in a form of a yellowish thick syrup. Total omega-3 content: 594 mg/g (86% yield). EPA content: 351 mg/g (86% yield). DHA content: 178 mg/g (85% yield). Zinc content was 11.7%.

### Example 6

### Use of Potassium Hydroxide

A potassium salt was prepared using 10.2 g (30 mmol) 40:20 EE (40 wt. % EPA ethyl ester and 20 wt. % DHA ethyl ester), 1.86 g ethanol, and 2.10 g (37.5 mmol) KOH in 2.10 g water. This refluxed for 1 h under nitrogen at 80-85° C. This was lyophilized and the resulting salt was pulverized into a beige powder. Total omega-3 content: 658 mg/g (97% yield). EPA content: 389 mg/g (97% yield). DHA content: 198 mg/g (97% yield). Potassium content was 11.9%.

### Example 7

### Use of Sodium Hydroxide

Sodium salt was prepared as in Example 6 except that 1.5 g (37.5 mmol) of NaOH in 2.25 mL (125 mmol) of water was added. The resulting sodium salt was in a form of a waxy yellow solid. Total omega-3 content: 654 mg/g (92% yield). EPA content: 388 mg/g (92% yield). DHA content: 195 mg/g (91% yield). Sodium content was 6.5%.

### Example 8

### Bioavailability Analysis

Ca-omega-3 salt (JW1378) used for the bioavailability study was prepared as described in Example 1 and the resulting salt contained 11.7% of calcium, 310.31 mg/g of EPA, 148.93 mg/g of DHA, and the total omega-3 as FFA was 526.16 mg/g. Mg-omega-3 Salt (JW1373) used for the bioavailability study was prepared following Example 4 and the resulting salt contained 2.72% of magnesium, 343.89 mg/g of EPA, 171.45 mg/g of DHA, and the total omega-3 as FFA was 598.20 mg/g.

C57 BL/6 mice were divided into 3 groups containing 10 animals in each group. After acclimation, treatment groups received fish oil preparations daily by oral gavage for 3 weeks, at a dose of 8 mg/day. Thus, mice in the control (ethyl ester), Ca-omega-3, and Mg-omega-3 groups received about 5.3, 4.2, and 4.8 mg omega-3 fatty acids daily. The dosage of omega-3 fatty acids was lower in the omega-3 salt groups compared to the fish oil control group based on the fatty analysis of the products (see Table 1). This dose range approximates a 1-gram per day dose in humans using typical scaling assumptions. The Ca- and Mg-omega-3 salts were suspended in glycerol by heating to 37° C. and sonication for 5-10 minutes. The Control group received vehicle (glycerol) spiked with fish oil, concentrate used to prepare the salts (40:20 ethyl ester). Fecal samples were collected weekly and pooled for each group. At the end of the study, blood was collected by heart puncture. Blood serum and red blood cells were isolated. Blood serum, red blood cells and fecal samples were analyzed for omega-3 fatty acid content.

The levels of EPA, DHA, and total omega-3 fatty acid of these products are shown in Table 5.

TABLE 5
Omega-3 Content of 40:20 Ethyl Ester, Ca-Omega-3 Salt,
and Mg-Omega-3 Salt used in the bioavailability study.

| | 40:20 Ethyl Ester | Ca-Omega-3 Salt | Mg-Omega-3 Salt |
|---|---|---|---|
| EPA (mg/g) | 388.1 | 310.3 | 343.9 |
| DHA (mg/g) | 188.7 | 148.9 | 171.5 |
| Total omega-3 | 660.5 | 526.2 | 598.2 |
| (mg/g)¹ | | | |
| Ca (weight %) | — | 11.7² | — |
| Mg (weight %) | | | 2.7³ |

¹Total omega-3 fatty acids includes EPA, DHA, and other omega-3 species (e.g., docosapentanoic acid, DPA).
²Theoretical is 6.2%. This was a lab sample with unreacted Ca oxide (or hydroxide) present.
³Theoretical is 3.8%.

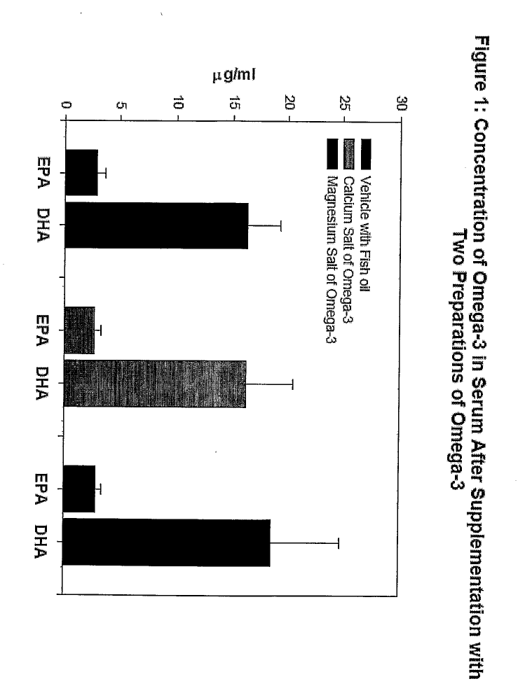

Ca- or Mg-omega-3 salt supplementation resulted in serum EPA and DHA content that were equivalent to that seen in the ethyl ester supplemented group (FIG. 1).

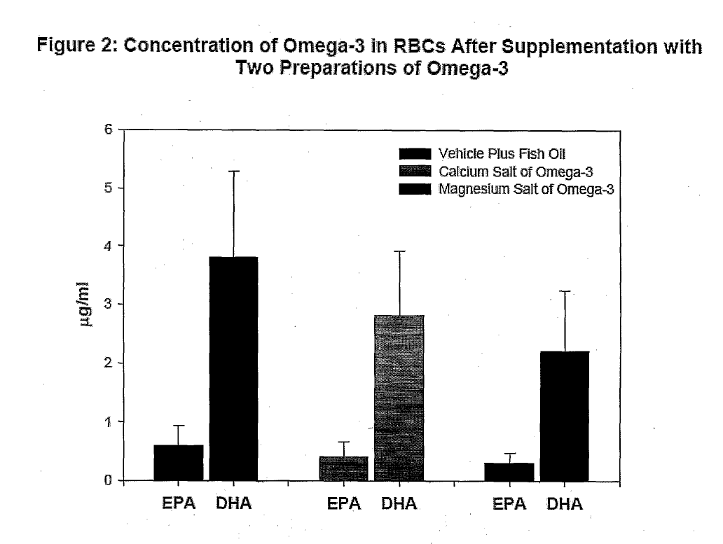

Ca-omega-3 salt supplementation resulted in incorporation of EPA and DHA into Red Blood cells to a similar extent as that seen in the control group. However, Mg-omega-3 salt supplementation resulted in slightly lower degree of EPA and DHA (p<0.05) (FIG. 2).

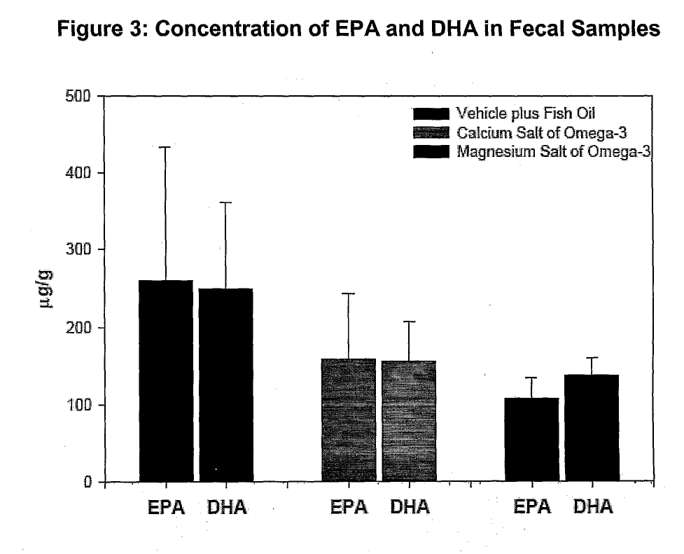

Fecal samples for each group were pooled for the 3-week period and compared (FIG. 3). Interestingly, there was a tendency for the Ca- and Mg-omega-3 salt products to result in lower fecal excretion of EPA and DHA than the ethyl ester oil, but only in the Mg-omega-3 salt group was this statistically significant.

In summary, it was found that the omega-3 salt products, especially the calcium versions, worked remarkably well, resulting in similar levels of serum and RBC omega-fatty acids as the oil control. This was true even though the dosage of omega-3 fatty acids in the omega-fatty salt groups were lower compared to that of the control. In addition, fecal excretion of omega-3 was not increased by the presence of calcium or magnesium. In fact, there was a tendency for less omega-3 fatty acids to be excreted.

### Example 9

A 290:235 triglyceride oil (e.g., an oil containing about 290 wt. % of EPA and about 235 wt. % DHA in their triglyceride forms) was converted into its various salts as described herein. Specifically, calcium salts were prepared from either CaCl₂ or CaO, and the potassium, magnesium, and zinc salts were prepared as described herein. The obtained salts were analyzed prior to being washed. The results are shown in Table 6.

TABLE 6
Analysis of Unwashed Salts from 290:235 TG oil

| | | Ca-omega-3 | | | | |
|---|---|---|---|---|---|---|
| | | Salt via | Ca-omega-3 | Mg-omega-3 | Zn-omega-3 | K-omega-3 |
| | | CaCl₂ | Salt via CaO | Salt | Salt | Salt |
| | 290:235 TG | (% yield) | (% yield) | (% yield) | (% yield) | (% yield) |
| EPA (mg/g) | 343 | 338 (99) | 329 (96) | 356 (104) | 273 (80) | 328 (96) |
| DHA (mg/g) | 274 | 266 (97) | 264 (96) | 285 (104) | 219 (80) | 262 (96) |
| Total omega-3 | 705 | 690 (98) | 678 (96) | 730 (104) | 562 (80) | 672 (95) |
| (mg/g)¹ | | | | | | |
| Mg | | | | 4.2% | | |
| Ca | | 6.6% | 6.6% | | | |
| Na | | 0.3% | | 0.5% | 4.3% | |
| K | | | | | | 10.6% |
| Zn | | | | | 9.0% | |
| Ash | | 9.5% | 8.6% | 6.7% | 19.4% | 13.2% |

Trace Metal Analysis (mg/kg)

| | | | | | |
|---|---|---|---|---|---|
| Aluminum | <5 | 60 | <5 | <5 | <5 |
| Antimony | <0.5 | <0.5 | <0.5 | <0.5 | <0.5 |
| Arsenic | <0.5 | <0.5 | <0.5 | 1.46 | <0.5 |
| Barium | <5 | <5 | <5 | <5 | <5 |
| Beryllium | <0.1 | <0.1 | <0.1 | <0.1 | <0.1 |
| Bismuth | <0.5 | <0.5 | <0.5 | <0.5 | <0.5 |
| Boron | <5 | <5 | <5 | <5 | 14 |
| Cadmium | <0.05 | <0.05 | <0.05 | 0.30 | <0.05 |
| Calcium | 66300 | 66300 | <50 | <50 | <50 |
| Chromium | <5 | <5 | <5 | <5 | <5 |
| Cobalt | <0.5 | <0.5 | <0.5 | <0.5 | <0.5 |
| Copper | <0.25 | 0.35 | <0.25 | 1.76 | <0.25 |
| Iron | <2.5 | 45.6 | <2.5 | <2.5 | <2.5 |
| Lead | <0.25 | <0.25 | <0.25 | <0.25 | <0.25 |
| Lithium | <0.5 | <0.5 | <0.5 | <0.5 | <0.5 |
| Magnesium | 21 | 297 | 41600 | 18 | <10 |
| Manganese | <5 | <5 | <5 | <5 | <5 |
| Mercury | <0.005 | <0.005 | <0.005 | <0.005 | <0.005 |
| Molybdenum | <0.5 | <0.5 | <0.5 | <0.5 | <0.5 |
| Nickel | <0.5 | <0.5 | <0.5 | <0.5 | <0.5 |
| Potassium | <25 | <25 | <25 | <25 | 106000 |
| Rubidium | <0.5 | <0.5 | <0.5 | <0.5 | 6.0 |
| Selenium | <5 | <5 | <5 | <5 | <5 |
| Silver | <0.5 | <0.5 | <0.5 | <0.5 | <0.5 |
| Sodium | 2700 | <50 | 4730 | 42900 | <50 |
| Strontium | 16.4 | 27.9 | <1 | <1 | <1 |
| Tellurium | <0.5 | <0.5 | <0.5 | <0.5 | <0.5 |
| Thallium | <0.5 | <0.5 | <0.5 | <0.5 | <0.5 |
| Tin | <0.5 | <0.5 | <0.5 | <0.5 | <0.5 |
| Uranium | <0.5 | <0.5 | <0.5 | <0.5 | <0.5 |
| Vanadium | <5 | <5 | <5 | <5 | <5 |
| Zinc | <5 | <5 | <5 | 89700 | <5 |
| Chloride | 2400 | <50 | 2700 | 36200 | <50 |

¹Total omega-3 fatty acids includes EPA, DHA, and other omega-3 species (e.g., docosapentanoic acid, DPA).

Certain salts were washed or extracted and then analyzed. Specifically, the calcium salt prepared from CaCl₂ was washed with water, and the calcium salt prepared from CaO was extracted with THF. The magnesium salt was washed with water, and the zinc salt was extracted with heptane and washed with water. These results are provided below in Table 7.

TABLE 7
Analysis of Washed or Extracted Salts from 290:235 TG oil

| | Ca-omega-3 | | | |
|---|---|---|---|---|
| | Salt Via | Ca-omega-3 | Mg-omega-3 | Zn-omega-3 |
| | CaCl₂ | Salt Via CaO | Salt | Salt |
| | (% yield) | (% yield) | (% yield) | (% yield) |
| EPA (mg/g) | 344 (100) | 322 (94) | 335 (98) | 310 (90) |
| DHA (mg/g) | 270 (99) | 254 (93) | 269 (98) | 244 (89) |
| Total omega-3 | 701 (99) | 660 (94) | 689 (98) | 633 (90) |
| (mg/g)¹ | | | | |
| Mg | | | 4.3% | |
| Ca | 6.6% | 6.8% | | |
| Na | 0.2% | | 0.6% | 5.5% |
| K | | | | |
| Zn | | | | 8.9% |
| Ash | 9.6% | 7.2% | 7.1% | 12.3% |

Trace Metal Analysis (mg/kg)

| | | | | |
|---|---|---|---|---|
| Aluminum | <5 | 9 | <5 | <5 |
| Antimony | <0.5 | <0.5 | <0.5 | <0.5 |
| Arsenic | <0.5 | <0.5 | <0.5 | 1.14 |
| Barium | <5 | <5 | <5 | <5 |
| Beryllium | <0.1 | <0.1 | <0.1 | <0.1 |
| Bismuth | <0.5 | <0.5 | <0.5 | <0.5 |
| Boron | <5 | <5 | <5 | <5 |
| Cadmium | <0.05 | <0.05 | <0.05 | <0.05 |
| Calcium | 65700 | 67900 | <50 | <50 |
| Chromium | <5 | <5 | <5 | <5 |
| Cobalt | <0.5 | <0.5 | <0.5 | <0.5 |
| Copper | 0.26 | <0.25 | <0.25 | 2.01 |
| Iron | <2.5 | 8.4 | <2.5 | <2.5 |
| Lead | <0.25 | <0.25 | <0.25 | <0.25 |
| Lithium | <0.5 | <0.5 | <0.5 | <0.5 |
| Magnesium | 20 | 14 | 42500 | 18 |
| Manganese | <5 | <5 | <5 | <5 |
| Mercury | <0.005 | <0.005 | <0.005 | <0.005 |
| Molybdenum | <0.5 | <0.5 | <0.5 | <0.5 |
| Nickel | <0.5 | <0.5 | <0.5 | 0.6 |
| Potassium | <25 | <25 | <25 | <25 |
| Rubidium | <0.5 | <0.5 | <0.5 | <0.5 |
| Selenium | <5 | <5 | <5 | <5 |
| Silver | <0.5 | <0.5 | <0.5 | <0.5 |
| Sodium | 1500 | <50 | 6020 | 5530 |
| Strontium | 16.0 | 17.8 | <1 | <1 |
| Tellurium | <0.5 | <0.5 | <0.5 | <0.5 |
| Thallium | <0.5 | <0.5 | <0.5 | <0.5 |
| Tin | <0.5 | <0.5 | <0.5 | <0.5 |
| Uranium | <0.5 | <0.5 | <0.5 | <0.5 |
| Vanadium | <5 | <5 | <5 | <5 |
| Zinc | <5 | <5 | <5 | 88500 |
| Chloride | 400 | <50 | 3800 | <50 |

¹Total omega-3 fatty acids includes EPA, DHA, and other omega-3 species (e.g., docosapentanoic acid, DPA).

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A composition, comprising at least one calcium salt of an omega-3 fatty acid, wherein the composition comprises at least about 30 weight % of one or more omega-3 fatty acid residues.

2. (canceled)

3. (canceled)

4. The composition of any of claim 1, wherein the one or more omega-3 fatty residues comprises at least about 75% by weight of the composition.

5. The composition of claim 1, wherein the composition is derived from microbial oil.

6. (canceled)

7. The composition of claim 1, wherein the composition is derived from fish oil.

8. (canceled)

9. (canceled)

10. The composition of claim 1, wherein the composition is not derived from a triglyceride oil.

11. The composition of claim 1, wherein the composition is derived from an esterified oil, a crude oil, a semi-refined oil, a refined oil, or a re-esterified oil.

12. (canceled)

13. The composition of claim 1, wherein the calcium salt of the omega-3 fatty acid is derived from linolenic acid, octadecatetraenoic acid, eicosapentaenoic acid (EPA), docosahexaenoic acid (DHA), docosapentaenoic acid (DPA), or a residue, derivatives, or mixture thereof.

14. The composition of claim 1, wherein the composition comprises residues of EPA and DHA in a weight % ratio of from about 60:0.3 or 0.8:60.

15. The composition of claim 1, wherein the composition comprises residues of EPA and DHA in a weight % ratio of from about 40:20.

16. The composition of claim 1, wherein the composition comprises a residue of EPA from about 30 to about 50 weight %.

17. The composition of claim 1, wherein the composition comprises a residue of DHA from about 10 to about 30 weight %.

18. (canceled)

19. The composition of claim 1, wherein the composition comprises less than about 10% by weight of conjugated linoleic acids.

20. (canceled)

21. The composition of claim 1, wherein the composition comprises calcium at from about 1 to about 15 weight %.

22. A composition, comprising at least one magnesium salt of an omega-3 fatty acid, wherein the composition comprises at least about 30 weight % of one or more omega-3 fatty acid residues.

23. (canceled)

24. (canceled)

25. The composition of claim 22, wherein the composition comprises at least about 75% by weight of one or more omega-3 fatty acid residues.

26. The composition of claim 22, wherein the composition is derived from microbial oil.

27. (canceled)

28. The composition of claim 22, wherein the composition is derived from fish oil.

29. (canceled)

30. (canceled)

31. The composition of claim 22, wherein the composition is not derived from a triglyceride oil.

32. The composition of claim 22, wherein the composition is derived from an esterified oil, a crude oil, a semi-refined oil, a refined oil, or a re-esterified oil.

33. (canceled)

34. The composition of claim 22, wherein the magnesium salt of the omega-3 fatty acid is derived from linolenic acid, octadecatetraenoic acid, eicosapentaenoic acid (EPA), docosahexaenoic acid (DHA), docosapentaenoic acid (DPA), or a residue, derivatives, or mixture thereof.

35. The composition of claim 22, wherein the composition comprises residues of EPA and DHA in a weight % ratio of from about 18:12, 5:25, 60:0.3, or 0.8:60.

36. The composition of claim 22, wherein the composition comprises residues of EPA and DHA in a weight % ratio of from about 40:20.

37. The composition of claim 22, wherein the composition comprises a residue of EPA from about 30 to about 50 weight %.

38. The composition of claim 22, wherein the composition comprises a residue of DHA from about 10 to about 30 weight %.

39. (canceled)

40. The composition of claim 22, wherein the composition comprises less than about 10% by weight of conjugated linoleic acids.

41. (canceled)

42. (canceled)

43. The composition of claim 22, wherein the composition comprises magnesium at from about 1 to about 15 weight %.

44. A composition, comprising at least one zinc salt of an omega-3 fatty acid, wherein the composition comprises at least about 30 weight % of one or more omega-3 fatty acid residues.

45. (canceled)

46. (canceled)

47. The composition of claim 44, wherein the composition comprises at least about 75% by weight of one or more omega-3 fatty acid residues.

48. The composition of claim 44, wherein the composition is derived from microbial oil.

49. (canceled)

50. The composition of any claim 44, wherein the composition is derived from fish oil.

51. (canceled)

52. (canceled)

53. The composition of claim 44, wherein the composition is not derived from a triglyceride oil.

54. The composition of claim 44, wherein the composition is derived from an esterified oil, a crude oil, a semi-refined oil, a refined oil, or a re-esterified oil.

55. (canceled)

56. The composition of claim 44, wherein the zinc salt of the omega-3 fatty acid is derived from linolenic acid, octadecatetraenoic acid, eicosapentaenoic acid (EPA), docosahexaenoic acid (DHA), docosapentaenoic acid (DPA), or a residue, derivatives, or mixture thereof.

57. The composition of claim 44, wherein the composition comprises residues of EPA and DHA in a weight % ratio of from about 18:12, 5:25, 60:0.3, or 0.8:60.

58. The composition of claim 44, wherein the composition comprises residues of EPA and DHA in a weight % ratio of from about 40:20.

59. The composition of claim 44, wherein the composition comprises a residue of EPA from about 30 to about 50 weight %.

60. The composition of claim 44, wherein the composition comprises a residue of DHA from about 10 to about 30 weight %.

61. (canceled)

62. The composition of any of claim 44, wherein the composition comprises less than about 10% by weight of conjugated linoleic acids.

63. (canceled)

64. The composition of claim 44, wherein the composition comprises zinc at from about 1 to about 15 weight %.

65. A composition, comprising at least one sodium salt, potassium salt, or a mixture thereof of an omega-3 fatty acid, wherein the composition comprises at least about 30 weight % of one or more omega-3 fatty acid residues.

66. (canceled)

67. (canceled)

68. The composition of claim 65, wherein the composition comprises at least about 75% by weight of one or more omega-3 fatty acid residues.

69. The composition of claim 65, wherein the composition is derived from microbial oil.

70. (canceled)

71. The composition of claim 65, wherein the composition is derived from fish oil.

72. (canceled)

73. (canceled)

74. The composition of claim 65, wherein the composition is not derived from a triglyceride oil.

75. The composition of claim 65, wherein the composition is derived from an esterified oil, a crude oil, a semi-refined oil, a refined oil, or a re-esterified oil.

76. (canceled)

77. The composition of claim 65, wherein the sodium salt, potassium salt, or mixture thereof of the omega-3 fatty acid is derived from linolenic acid, octadecatetraenoic acid, eicosapentaenoic acid (EPA), docosahexaenoic acid (DHA), docosapentaenoic acid (DPA), or a residue, derivatives, or mixture thereof.

78. The composition of claim 65, wherein the composition comprises residues of EPA and DHA in a weight % ratio of from about 18:12, 5:25, 60:0.3, or 0.8:60.

79. The composition of claim 65, wherein the composition comprises residues of EPA and DHA in a weight % ratio of from about 40:20.

80. The composition of claim 65, wherein the composition comprises a residue of EPA from about 30 to about 50 weight %.

81. The composition of claim 65, wherein the composition comprises a residue of DHA from about 10 to about 30 weight %.

82. (canceled)

83. The composition of claim 65, wherein the composition comprises less than about 10% by weight of conjugated linoleic acids.

84. The composition of claim 65, wherein the composition comprises sodium or potassium at from about 1 to about 15 weight %.

85. A composition, comprising at least two salts chosen from a calcium salt of an omega-3 fatty acid, a magnesium salt of an omega-3 fatty acid, a sodium salt of an omega-3 fatty acid, a potassium salt of an omega-3 fatty acid, and a zinc salt of an omega-3 fatty acid.

86-113. (canceled)
